(19) United States
(12) Patent Application Publication
(10) Pub. No.: US 2013/0130385 A1
(43) Pub. Date: May 23, 2013

(54) SURFACE MARKERS AND USES THEREOF FOR RAPID STABLE CELL LINE GENERATION AND GENE AMPLIFICATION

(71) Applicant: Hua Tu, San Carlos, CA (US)

(72) Inventor: Hua Tu, San Carlos, CA (US)

(21) Appl. No.: 13/696,586

(22) PCT Filed: May 6, 2011
(86) PCT No.: PCT/US2011/035656

§ 371 Date: Feb. 4, 2013

Related U.S. Application Data
(60) Provisional application No. 61/332,583, filed on May 7, 2010.

(51) Int. Cl. C12N 15/65

(52) U.S. Cl. CPC C12N 15/65
USPC 435/455; 435/320.1

(57) ABSTRACT

The present invention provides methods of producing recombinant cells, methods of large scale production of a gene expression product (such as protein), and methods of establishing a stable cell line using the surface markers. Also provided are expression vectors encoding the surface markers and cells comprising the expression vectors. Further provided are gene expression products (such as proteins) and cells obtained using methods described herein, as well as kits useful for carrying out methods described herein.

### RELATED APPLICATIONS

This application claims priority benefit to U.S. Provisional Application No. 61/332,583, filed on May 7, 2010, the content of which is incorporated herein in its entirety.

### TECHNICAL FIELD

The present invention relates to surface markers and uses thereof for the production of recombinant cells, large scale protein production and establishment of stable cell lines.

### BACKGROUND

Protein productions in preclinical and clinical settings often require multigram quantities. Industrial applications using such proteins require even greater quantities. To meet such requirements, it is important that cells used for protein production express the proteins at high levels.

One strategy employed in increasing protein expression in host cells is to effectively increase the gene dosage in a transfected host cell. This is most commonly achieved by using cell lines deficient in enzymes such as DHFR (dihydrofolate reductase) or GS (glutamine synthetase). Expression vectors containing genes encoding those enzymes are then introduced into the cells, and cell having a desired expression levels can then be selected using agents such as methotrexate (MTX), which inhibits DHFR, and methionine sulfoxamine (MSX), which inhibit GS. Gene amplification can be achieved by growing the transfectants in progressively increasing concentrations of MTX or MSX.

While gene amplification methods using DHFR and GS can result in higher levels of expression, it has many drawbacks. First, cell lines that have mutations in the genes encoding the selective enzymes are generally used for gene amplification. In the case of DHFR, both chromosomal copies need to be mutated and, consequently, these cell lines can be less robust than wild type cells. This can ultimately lead to cells which secret lower net amounts of the protein of interest as compared to more robust cells that thrive and are stable. In the case of GS, the lymphoid cell line NSO is naturally GS negative, but CHO-K1, another commonly used cell line, is GS positive and requires selection directly for MSX-resistant transfectants.

Second, selective pressure for selecting cells with high gene copy and gene is frequently required. When the selective pressure is removed, expression may become unstable or even extinguished. Only a small number of initial transformants can provide high and stable long-term expression, which are hard to identify from among a large population of candidates.

Finally, because of the multiple selection steps needed for the current methods, the current methods are time consuming and costly.

There is thus a continued need for new methods for selecting cells with high levels of gene amplification and protein expression.

The disclosures of all publications, patents, patent applications and published patent applications referred to herein are hereby incorporated herein by reference in their entirety.

### BRIEF SUMMARY OF THE INVENTION

The present invention in one aspect provides methods of producing a recombinant cell comprising a gene of interest. “Gene of interest” used throughout the present application includes, for example, a nucleic acid sequence such as cDNA encoding a product (such as protein) of interest. In some embodiments, the method comprises: exposing a population of host cells comprising (for example comprising a vector comprising): i) a first nucleic acid sequence comprising the gene of interest, and ii) a second nucleic acid sequence comprising a coding sequence for a surface marker to a separation means that recognizes the surface marker, wherein cells recognized by the separation means can be separated from the rest of the cells. In some embodiments, there is provided a method of producing a recombinant cell comprising a gene of interest (for example a nucleic acid sequence such as cDNA encoding a protein of interest), comprising: exposing a population of host cells comprising a vector comprising: i) a first nucleic acid sequence comprising the gene of interest, and ii) a second nucleic acid sequence comprising a coding sequence for a surface marker to a separation means that recognizes the surface marker, wherein cells recognized by the separation means can be separated from the rest of the cells.

In some embodiments, there is provided a method of large scale production of a gene expression product from a gene of interest (for example a nucleic acid sequence such as cDNA encoding a protein of interest), comprising exposing a population of host cells comprising (for example comprising a vector comprising): i) a first nucleic acid sequence comprising the gene of interest, and ii) a second nucleic acid sequence comprising a coding sequence for a surface marker to a separation means that recognizes the surface marker, wherein cells recognized by the separation means can be separated from the rest of the cells. In some embodiments, there is provided a method of large scale production of a gene expression product from a gene of interest (for example a nucleic acid sequence such as cDNA encoding a protein of interest), comprising exposing a population of host cells comprising a vector comprising: i) a first nucleic acid sequence comprising the gene of interest, and ii) a second nucleic acid sequence comprising a coding sequence for a surface marker to a separation means that recognizes the surface marker, wherein cells recognized by the separation means can be separated from the rest of the cells.

In some embodiments, there is provided a method of establishing a stable cell line for expressing a protein of interest from a gene of interest (for example a nucleic acid sequence such as cDNA encoding the protein of interest), comprising exposing a population of host cells comprising (for example comprising a vector comprising): i) a first nucleic acid sequence comprising the gene of interest, and ii) a second nucleic acid sequence comprising a coding sequence for a surface marker to a separation means that recognizes the surface marker, wherein cells recognized by the separation means can be separated from the rest of the cells. In some embodiments, there is provided a method of establishing a stable cell line for expressing a protein of interest from a gene of interest (for example a nucleic acid sequence such as cDNA encoding the protein of interest), comprising exposing a population of host cells comprising a vector comprising: i) a first nucleic acid sequence comprising the gene of interest, and ii) a second nucleic acid sequence comprising a coding sequence for a surface marker to a separation means that recognizes the surface marker, wherein cells recognized by the separation means can be separated from the rest of the cells.

In some embodiments, there is provided a method of obtaining a recombinant cell having a high expression level for expressing a gene of interest (for example a nucleic acid sequence such as cDNA encoding a protein of interest) from a population of host cells comprising (for example comprising a vector comprising): i) a first nucleic acid sequence comprising the gene of interest, and ii) a second nucleic acid sequence comprising a coding sequence for a surface marker, comprising exposing the cells to a separation means that recognizes the surface marker, wherein cells having a high expression level can be separated from the rest of the cells. In some embodiments, there is provided a method of obtaining a recombinant cell having a high expression level for expressing a gene of interest (for example a nucleic acid sequence such as cDNA encoding a protein of interest) from a population of host cells comprising a vector comprising: i) a first nucleic acid sequence comprising the gene of interest, and ii) a second nucleic acid sequence comprising a coding sequence for a surface marker, the method comprising exposing the cells to a separation means that recognizes the surface marker, wherein cells having a high expression level can be separated from the rest of the cells.

In some embodiments, the surface marker comprises a tag sequence and a transmembrane domain. In some embodiments, the surface marker comprises a tag sequence and a membrane anchoring region. In some embodiments, the tag sequence is less than about 100 amino acids. “Tag sequence” used herein refers to the amino acid sequence on the extracellular domain of the surface marker that allows recognition by the separation means. Suitable tag sequences include, for example, FLAG, His, HA, myc, and V5.

Thus, for example, in some embodiments, there is provided a method of producing a recombinant cell comprising a gene of interest (for example a nucleic acid sequence such as cDNA encoding a protein of interest), comprising: exposing a population of host cells comprising a vector comprising: i) a first nucleic acid sequence comprising the gene of interest, and ii) a second nucleic acid sequence comprising a coding sequence for a surface marker to a separation means that recognizes the surface marker, wherein the surface marker comprises a tag sequence that is less than about 100 amino acids, wherein cells recognized by the separation means can be separated from the rest of the cells. In some embodiments, there is provided a method of large scale production of a gene expression product from a gene of interest (for example a nucleic acid sequence such as cDNA encoding a protein of interest), comprising exposing a population of host cells comprising a vector comprising: i) a first nucleic acid sequence comprising the gene of interest, and ii) a second nucleic acid sequence comprising a coding sequence for a surface marker to a separation means that recognizes the surface marker, wherein the surface marker comprises a tag sequence that is less than about 100 amino acids, and wherein cells recognized by the separation means can be separated from the rest of the cells. In some embodiments, there is provided a method of establishing a stable cell line for expressing a protein of interest from a gene of interest (for example a nucleic acid sequence such as cDNA encoding the protein of interest), comprising exposing a population of host cells comprising a vector comprising: i) a first nucleic acid sequence comprising the gene of interest, and ii) a second nucleic acid sequence comprising a coding sequence for a surface marker to a separation means that recognizes the surface marker, wherein the surface marker comprises a tag sequence that is less than about 100 amino acids, and wherein cells recognized by the separation means can be separated from the rest of the cells. In some embodiments, there is provided a method of obtaining a recombinant cell having a high expression level for expressing a gene of interest (for example a nucleic acid sequence such as cDNA encoding a protein of interest) from a population of host cells comprising a vector comprising: i) a first nucleic acid sequence comprising the gene of interest, and ii) a second nucleic acid sequence comprising a coding sequence for a surface marker, the method comprising exposing the cells to a separation means that recognizes the surface marker, wherein the surface marker comprises a tag sequence that is less than about 100 amino acids, and wherein cells having a high expression level can be separated from the rest of the cells.

In some embodiments, the surface marker comprises one or more of the following: FLAG, His, HA, myc, and/or V5. In some embodiments, there is provided a method of producing a recombinant cell comprising a gene of interest (for example a nucleic acid sequence such as cDNA encoding a protein of interest), comprising: exposing a population of host cells comprising a vector comprising: i) a first nucleic acid sequence comprising the gene of interest, and ii) a second nucleic acid sequence comprising a coding sequence for a surface marker to a separation means that recognizes the surface marker, wherein the surface marker comprises one or more of the following: FLAG, His, HA, myc, and/or V5, wherein cells recognized by the separation means can be separated from the rest of the cells. In some embodiments, there is provided a method of large scale production of a gene expression product from a gene of interest (for example a nucleic acid sequence such as cDNA encoding a protein of interest), comprising exposing a population of host cells comprising a vector comprising: i) a first nucleic acid sequence comprising the gene of interest, and ii) a second nucleic acid sequence comprising a coding sequence for a surface marker to a separation means that recognizes the surface marker, wherein the surface marker comprises one or more of the following: FLAG, His, HA, myc, and/or V5, and wherein cells recognized by the separation means can be separated from the rest of the cells. In some embodiments, there is provided a method of establishing a stable cell line for expressing a protein of interest from a gene of interest (for example a nucleic acid sequence such as cDNA encoding the protein of interest), comprising exposing a population of host cells comprising a vector comprising: i) a first nucleic acid sequence comprising the gene of interest, and ii) a second nucleic acid sequence comprising a coding sequence for a surface marker to a separation means that recognizes the surface marker, wherein the surface marker comprises one or more of the following: FLAG, His, HA, myc, and/or V5, and wherein cells recognized by the separation means can be separated from the rest of the cells. In some embodiments, there is provided a method of obtaining a recombinant cell having a high expression level for expressing a gene of interest (for example a nucleic acid sequence such as cDNA encoding a protein of interest) from a population of host cells comprising a vector comprising: i) a first nucleic acid sequence comprising the gene of interest, and ii) a second nucleic acid sequence comprising a coding sequence for a surface marker, the method comprising exposing the cells to a separation means that recognizes the surface marker, the surface marker comprises one or more of the following: FLAG, His, HA, myc, and/or V5, and wherein cells having a high expression level can be separated from the rest of the cells.

In some embodiments, the surface marker comprises one or more copies of FLAG (such as N-terminal FLAG). In some embodiments, there is provided a method of producing a recombinant cell comprising a gene of interest (for example a nucleic acid sequence such as cDNA encoding a protein of interest), comprising: exposing a population of host cells comprising a vector comprising: i) a first nucleic acid sequence comprising the gene of interest, and ii) a second nucleic acid sequence comprising a coding sequence for a surface marker to a separation means (for example an anti-FLAG antibody, such as a calcium-dependent anti-FLAG antibody) that recognizes the surface marker, wherein the surface marker comprises one or more copies of FLAG (such as N-terminal FLAG), wherein cells recognized by the separation means can be separated from the rest of the cells. In some embodiments, there is provided a method of large scale production of a gene expression product from a gene of interest (for example a nucleic acid sequence such as cDNA encoding a protein of interest), comprising exposing a population of host cells comprising a vector comprising: i) a first nucleic acid sequence comprising the gene of interest, and ii) a second nucleic acid sequence comprising a coding sequence for a surface marker to a separation means (for example an anti-FLAG antibody, such as a calcium-dependent anti-FLAG antibody) that recognizes the surface marker, wherein the surface marker comprises one or more copies of FLAG (such as N-terminal FLAG), and wherein cells recognized by the separation means can be separated from the rest of the cells. In some embodiments, there is provided a method of establishing a stable cell line for expressing a protein of interest from a gene of interest (for example a nucleic acid sequence such as cDNA encoding the protein of interest), comprising exposing a population of host cells comprising a vector comprising: i) a first nucleic acid sequence comprising the gene of interest, and ii) a second nucleic acid sequence comprising a coding sequence for a surface marker to a separation means (for example an anti-FLAG antibody, such as a calcium-dependent anti-FLAG antibody) that recognizes the surface marker, wherein the surface marker comprises one or more copies of FLAG (such as N-terminal FLAG), and wherein cells recognized by the separation means can be separated from the rest of the cells. In some embodiments, there is provided a method of obtaining a recombinant cell having a high expression level for expressing a gene of interest (for example a nucleic acid sequence such as cDNA encoding a protein of interest) from a population of host cells comprising a vector comprising: i) a first nucleic acid sequence comprising the gene of interest, and ii) a second nucleic acid sequence comprising a coding sequence for a surface marker, the method comprising exposing the cells to a separation means (for example an anti-FLAG antibody, such as a calcium-dependent anti-FLAG antibody) that recognizes the surface marker, the surface marker comprises one or more copies of FLAG (such as N-terminal FLAG), and wherein cells having a high expression level can be separated from the rest of the cells. In some embodiments, the method further comprises releasing the cells from the separation means. For example, when the anti-FLAG antibody is calcium dependent, the method can comprise adding a chelating agent (such as EDTA) to the cells to disrupt the binding of the cells to the separation means.

In some embodiments, the surface marker is RUM 873 or RUM 879. In some embodiments, there is provided a method of producing a recombinant cell comprising a gene of interest (for example a nucleic acid sequence such as cDNA encoding a protein of interest), comprising: exposing a population of host cells comprising a vector comprising: i) a first nucleic acid sequence comprising the gene of interest, and ii) a second nucleic acid sequence comprising a coding sequence for RUM 873 or RUM 879 to a separation means (for example an anti-FLAG antibody, such as a calcium-dependent anti-FLAG antibody) that recognizes the RUM 873 or RUM 879, wherein cells recognized by the separation means can be separated from the rest of the cells. In some embodiments, there is provided a method of large scale production of a gene expression product from a gene of interest (for example a nucleic acid sequence such as cDNA encoding a protein of interest), comprising exposing a population of host cells comprising a vector comprising: i) a first nucleic acid sequence comprising the gene of interest, and ii) a second nucleic acid sequence comprising a coding sequence for RUM 873 or RUM 879 to a separation means (for example an anti-FLAG antibody, such as a calcium-dependent anti-FLAG antibody) that recognizes the RUM 873 or RUM 879, and wherein cells recognized by the separation means can be separated from the rest of the cells. In some embodiments, there is provided a method of establishing a stable cell line for expressing a protein of interest from a gene of interest (for example a nucleic acid sequence such as cDNA encoding the protein of interest), comprising exposing a population of host cells comprising a vector comprising: i) a first nucleic acid sequence comprising the gene of interest, and ii) a second nucleic acid sequence comprising a coding sequence for RUM 873 or RUM 879 to a separation means (for example an anti-FLAG antibody, such as a calcium-dependent anti-FLAG antibody) that recognizes the RUM 873 or RUM 879, and wherein cells recognized by the separation means can be separated from the rest of the cells. In some embodiments, there is provided a method of obtaining a recombinant cell having a high expression level for expressing a gene of interest (for example a nucleic acid sequence such as cDNA encoding a protein of interest) from a population of host cells comprising a vector comprising: i) a first nucleic acid sequence comprising the gene of interest, and ii) a second nucleic acid sequence comprising a coding sequence for RUM 873 or RUM 879, the method comprising exposing the cells to a separation means (for example an anti-FLAG antibody, such as a calcium-dependent anti-FLAG antibody) that recognizes the RUM 873 or RUM 879, and wherein cells having a high expression level can be separated from the rest of the cells. In some embodiments, the method further comprises releasing the cells from the separation means. For example, when the anti-FLAG antibody is calcium dependent, the method can comprise adding a chelating agent (such as EDTA) to the cells to disrupt the binding of the cells to the separation means.

In some embodiments, the expression vector has a gene map that is the same or similar gene map as p632 (or pDual-Selection). In some embodiments, the expression vector is p632 (or pDual-Selection). In some embodiments, there is provided a method of producing a recombinant cell comprising a gene of interest (for example a nucleic acid sequence such as cDNA encoding a protein of interest), comprising: exposing a population of host cells comprising a vector having a gene map that is the same or similar gene map as p632 (such as p632 or pDual-Selection) to a separation means (for example an anti-FLAG antibody, such as a calcium-dependent anti-FLAG antibody) that recognizes the surface marker encoded by the vector, wherein cells recognized by the separation means can be separated from the rest of the cells. In some embodiments, there is provided a method of large scale production of a gene expression product from a gene of interest (for example a nucleic acid sequence such as cDNA encoding a protein of interest), comprising exposing a population of host cells comprising a vector having a gene map that is the same or similar gene map as p632 (such as p632 or pDual-Selection) to a separation means (for example an anti-FLAG antibody, such as a calcium-dependent anti-FLAG antibody) that recognizes the surface marker encoded by the vector, and wherein cells recognized by the separation means can be separated from the rest of the cells. In some embodiments, there is provided a method of establishing a stable cell line for expressing a protein of interest from a gene of interest (for example a nucleic acid sequence such as cDNA encoding the protein of interest), comprising exposing a population of host cells comprising a vector having a gene map that is the same or similar gene map as p632 (such as p632 or pDual-Selection) to a separation means (for example an anti-FLAG antibody, such as a calcium-dependent anti-FLAG antibody) that recognizes the surface marker encoded by the vector, and wherein cells recognized by the separation means can be separated from the rest of the cells. In some embodiments, there is provided a method of obtaining a recombinant cell having a high expression level for expressing a gene of interest (for example a nucleic acid sequence such as cDNA encoding a protein of interest) from a population of host cells comprising a vector having a gene map that is the same or similar gene map as p632 (such as p632 or pDual-Selection), the method comprising exposing the cells to a separation means (for example an anti-FLAG antibody, such as a calcium-dependent anti-FLAG antibody) that recognizes the surface marker encoded by the vector, wherein cells having a high expression level can be separated from the rest of the cells. In some embodiments, the method further comprises releasing the cells from the separation means. For example, when the anti-FLAG antibody is calcium dependent, the method can comprise adding a chelating agent (such as EDTA) to the cells to disrupt the binding of the cells to the separation means.

In some embodiments, the separation means is a ligand specifically recognizing the surface marker. In some embodiments, the ligand is attached to a magnetic bead. In some embodiments, the ligand is attached to a label. In some embodiments, the ligand is attached to a solid support. Thus, for example, in some embodiments, there is provided a method of producing a recombinant cell comprising a gene of interest (for example a nucleic acid sequence such as cDNA encoding a protein of interest), comprising: exposing a population of host cells comprising a vector comprising: i) a first nucleic acid sequence comprising the gene of interest, and ii) a second nucleic acid sequence comprising a coding sequence for a surface marker to a ligand (for example a ligand coating a magnetic bead) that recognizes the surface marker, wherein cells recognized by the ligand can be separated from the rest of the cells. In some embodiments, there is provided a method of producing a recombinant cell comprising a gene of interest (for example a nucleic acid sequence such as cDNA encoding a protein of interest), comprising: exposing a population of host cells comprising a vector comprising: i) a first nucleic acid sequence comprising the gene of interest, and ii) a second nucleic acid sequence comprising a coding sequence for a surface marker to a ligand (for example a ligand coating a magnetic bead) that recognizes the surface marker, wherein the surface marker comprises a tag sequence that is less than about 100 amino acids, wherein cells recognized by the ligand can be separated from the rest of the cells. In some embodiments, there is provided a method of obtaining a recombinant cell having a high expression level for expressing a gene of interest (for example a nucleic acid sequence such as cDNA encoding a protein of interest) from a population of host cells comprising a vector comprising: i) a first nucleic acid sequence comprising the gene of interest, and ii) a second nucleic acid sequence comprising a coding sequence for a surface marker, the method comprising exposing the cells to a ligand (for example a ligand coating a magnetic bead) that recognizes the surface marker, wherein the surface marker comprises a tag sequence that is less than about 100 amino acids, and wherein cells having a high expression level can be separated from the rest of the cells. In some embodiments, the surface marker comprises one or more of the following: FLAG, His, HA, myc, and/or V5. In some embodiments, the surface marker is RUM 873 or RUM 879. In some embodiments, the expression vector has a gene map that is the same or similar gene map as p632 (or pDual-Selection). In some embodiments, the expression vector is p632 (or pDual-Selection).

In some embodiments, the method further comprises separating cells recognized by the separation means from the rest of the cells. In some embodiments, the method further comprises culturing the host cells for at least one day (such as at least any of two, three, four, or five days) prior to exposing the cells to a separation means. In some embodiments, the method further comprises introducing the first and second nucleic acid sequences into the cells. In some embodiments, the cells are exposed to a separation means within about any of 24 hours, 12 hours, 8 hours, 4 hours, 3 hours, or less after the introduction of the nucleic acid sequences into the cells. In some embodiments, the method further comprises introducing the first and second nucleic acid sequences into the cells.

Thus, for example, in some embodiments, there is provided a method of producing a recombinant cell comprising a gene of interest (for example a nucleic acid sequence such as cDNA encoding a protein of interest), comprising: a) culturing (such as culturing for at least any of one, two, three, four, or five days) a population of host cells comprising a vector comprising: i) a first nucleic acid sequence comprising the gene of interest, and ii) a second nucleic acid sequence comprising a coding sequence for a surface marker, b) exposing said cultured cells to a separation means that recognizes the surface marker, wherein cells recognized by the separation means can be separated from the rest of the cells. In some embodiments, there is provided a method of producing a recombinant cell comprising a gene of interest (for example a nucleic acid sequence such as cDNA encoding a protein of interest), comprising: a) culturing (such as culturing for at least any of one, two, three, four, or five days) a population of host cells comprising a vector comprising: i) a first nucleic acid sequence comprising the gene of interest, and ii) a second nucleic acid sequence comprising a coding sequence for a surface marker, b) exposing said cultured cells to a separation means that recognizes the surface marker, wherein the surface marker comprises a tag sequence that is less than about 100 amino acids, and wherein cells recognized by the separation means can be separated from the rest of the cells. In some embodiments, there is provided a method of producing a recombinant cell comprising a gene of interest (for example a nucleic acid sequence such as cDNA encoding a protein of interest), comprising: a) culturing (such as culturing for at least any of one, two, three, four, or five days) a population of host cells comprising a vector comprising: i) a first nucleic acid sequence comprising the gene of interest, and ii) a second nucleic acid sequence comprising a coding sequence for a surface marker, b) exposing said cultured cells to a ligand (for example a ligand coating a magnetic bead) that recognizes the surface marker, wherein the surface marker comprises a tag sequence that is less than about 100 amino acids, and wherein cells recognized by the ligand can be separated from the rest of the cells. In some embodiments, there is provided a method of obtaining a recombinant cell having a high expression level for expressing a gene of interest (for example a nucleic acid sequence such as a cDNA encoding a protein of interest) from a population of cells comprising a vector comprising: i) a first nucleic acid sequence comprising the gene of interest, and ii) a second nucleic acid sequence comprising a coding sequence for a surface marker, the method comprising: a) culturing the cells (such as culturing for at least any of one, two, three, four, or five days), b) exposing said cultured cells to a ligand (for example a ligand coating a magnetic bead) that recognizes the surface marker, wherein the surface marker comprises a tag sequence that is less than about 100 amino acids, and wherein cells recognized by the ligand can be separated from the rest of the cells. In some embodiments, the surface marker comprises one or more of the following: FLAG, His, HA, myc, and/or V5. In some embodiments, the surface marker is RUM 873 or RUM 879. In some embodiments, the expression vector has a gene map that is the same or similar gene map as p632 (or pDual-Selection). In some embodiments, the expression vector is p632 (or pDual-Selection).

In some embodiments, the first and second nucleic acid sequences are on the same expression vector. In some embodiments, the first and second nucleic acid sequences are on different expression vectors.

In some embodiments, the method further comprises washing the cells exposed to the separation means. In some embodiments, the method further comprises releasing the cells from the separation means. In some embodiments, the method further comprises harvesting the cells produced. For example, in some embodiments, there is provided a method of producing a recombinant cell comprising a gene of interest (for example a nucleic acid sequence such as cDNA encoding a protein of interest), comprising: exposing a population of host cells comprising a vector comprising: i) a first nucleic acid sequence comprising the gene of interest, and ii) a second nucleic acid sequence comprising a coding sequence for a surface marker to a ligand (for example a ligand coating a magnetic bead) that recognizes the surface marker, b) exposing the cells to a blocking agent that competes for the binding to the surface marker, and c) separating cells bound to the ligand from the rest of the cells. In some embodiments, there is provided a method of producing a recombinant cell comprising a gene of interest (for example a nucleic acid sequence such as cDNA encoding a protein of interest), comprising: exposing a population of host cells comprising a vector comprising a vector comprising: i) a first nucleic acid sequence comprising the gene of interest, and ii) a second nucleic acid sequence comprising a coding sequence for a surface marker to a ligand (for example a ligand coating a magnetic bead) that recognizes the surface marker, wherein the surface marker comprises a tag sequence that is less than about 100 amino acids, b) exposing the cells to a blocking agent that competes for the binding to the surface marker, and c) separating cells bound to the ligand from the rest of the cells. In some embodiments, there is provided a method of obtaining a recombinant cell having a high expression level for expressing a gene of interest (for example a nucleic acid sequence such as a cDNA encoding a protein of interest) from a population of host cells comprising a vector comprising a vector comprising: i) a first nucleic acid sequence comprising the gene of interest, and ii) a second nucleic acid sequence comprising a coding sequence for a surface marker, comprising a) exposing the cells to a ligand (for example a ligand coating a magnetic bead) that recognizes the surface marker, wherein the surface marker comprises a tag sequence that is less than about 100 amino acids, b) exposing the cells to a blocking agent that competes for the binding to the surface marker, and c) separating cells bound to the ligand from the rest of the cells. In some embodiments, the surface marker comprises one or more of the following: FLAG, His, HA, myc, and/or V5. In some embodiments, the surface marker is RUM 873 or RUM 879. In some embodiments, the expression vector has a gene map that is the same or similar gene map as p632 (or pDual-Selection). In some embodiments, the expression vector is p632 (or pDual-Selection).

In some embodiments, the cells further comprise a third nucleic acid sequence comprising a coding sequence for a second selectable marker that is different from the surface marker. In some embodiments, the method further comprises selecting cells based on the second selectable marker. The selection can be carried out before, during, or after exposing the cells to the separation means. Thus, for example, in some embodiments, there is provided a method of producing a recombinant cell comprising a gene of interest, comprising: exposing a population of host cells comprising (for example comprising a vector comprising: i) a first nucleic acid sequence comprising a gene of interest, ii) a second nucleic acid sequence comprising a coding sequence for a surface marker; and iii) a third nucleic acid sequence comprising a coding sequence for a selectable marker that is different from the surface marker to a separation means that recognizes the surface marker, wherein cells recognized by the separation means can be separated from the rest of the cells, wherein the method further comprises selecting cells based on the second selectable marker. In some embodiments, the selection based on the second selectable marker is carried out prior to exposing the cells to the separation means. In some embodiments, the selection based on the selectable marker is carried out during exposing the cells to the separation means. In some embodiments, the selection based on the selectable marker is carried out after exposing the cells to the separation means. In some embodiments, the surface marker comprises a tag sequence that is less than about 100 amino acids. In some embodiments, the surface marker comprises one or more of the following: FLAG, His, HA, myc, and/or V5. In some embodiments, the surface marker is RUM 873 or RUM 879. In some embodiments, the expression vector has a gene map that is the same or similar gene map as p632 (or pDual-Selection). In some embodiments, the expression vector is p632 (or pDual-Selection).

In some embodiments, more than one surface markers is used, and the more than one surface markers are particularly useful for methods of producing multimeric proteins. For example, in some embodiments, there is provided a method of producing a multimeric protein (such as an antibody), comprising: a) exposing a population of cells to a separation means, wherein the cells comprise: 1) a first expression vector comprising: i) a first nucleic acid sequence comprising a first gene of interest (such as a nucleic acid sequence encoding an antibody light chain), and ii) a second nucleic acid sequence comprising a coding sequence for a first surface marker; and 2) a second expression vector comprising: i) a second nucleic acid sequence comprising a second gene of interest (such as a nucleic acid sequence encoding an antibody heavy chain), and iv) a fourth nucleic acid sequence comprising a coding sequence for a second surface marker; wherein the first and second surface markers form a heterodimer on the cell surface that is recognizable by the separation means, and wherein the first and the second gene of interest produce polypeptides that form the multimeric protein; and b) separating cells recognized by the separation means from the rest of the cells. In some embodiments, there is provided a method of producing a multimeric protein (such as an antibody), comprising: a) exposing a population of cells to a separation means, wherein the cells comprise: 1) a first expression vector comprising: i) a first nucleic acid sequence comprising a first gene of interest (such as a nucleic acid encoding an antibody light chain), and ii) a second nucleic acid sequence comprising a coding sequence for a first surface marker; and 2) a second expression vector comprising: i) a second nucleic acid sequence comprising a second gene of interest (such as a nucleic acid encoding an antibody heavy chain), and iv) a fourth nucleic acid sequence comprising a coding sequence for a second surface marker; wherein the first and second surface markers each comprises a tag sequence that is less than about 100 amino acids, wherein the first and second surface markers form a heterodimer on the cell surface that is recognizable by the separation means, and wherein the first and the second gene of interest produce polypeptides that form the multimeric protein; and b) separating cells recognized by the separation means from the rest of the cells. In some embodiments, the surface marker comprises one or more of the following: FLAG, His, HA, myc, and/or V5. In some embodiments, the surface marker is RUM 873 or RUM 879. In some embodiments, the expression vector has a gene map that is the same or similar gene map as p632 (or pDual-Selection). In some embodiments, the expression vector is p632 (or pDual-Selection).

In another aspect, there is provided an expression vector comprising: i) a first cistron comprising a first promoter and a gene of interest, ii) a second cistron comprising a second promoter and a coding sequence for a surface marker, wherein the surface marker comprises a tag sequence and a transmembrane domain or a membrane anchoring region. In some embodiments, the first and second cistrons are positioned head to tail on the expression vector. In some embodiments, the first and second cistron are positioned tail to tail on the expression vector. In some embodiments, the first and second cistrons are positioned head to head on the expression vector.

In some embodiments, the tag sequence described herein is not scFv, CD4, MHC class I molecule H-2k^{k}, and human low affinity nerve growth factor receptor (LNGFR). In some embodiments, the tag sequence is less than about 100 amino acids. In some embodiments, the tag sequence comprises one or more of the following: FLAG, His, HA, myc, and V5. Other peptide epitopes or tags are also contemplated.

In some embodiments, the expression vector further comprises a third cistron comprising a third promoter and a coding sequence for a selectable marker that is different from the surface marker. In some embodiments, the first cistron is positioned between the second and third cistrons.

Also provided are cells comprising the nucleic acids or expression vectors described herein and uses thereof; cells produced by methods described herein and uses thereof; gene expression products (such as proteins, for example antibodies) produced by methods described herein and uses thereof; and kits that are useful for methods described herein.

### BRIEF DESCRIPTION OF FIGURES

FIG. 1 provides the protein sequence of RUM 873. Amino acids 20 and 21 correspond to restriction enzyme site for PstI.

FIG. 2 provides the protein sequence of RUM 879.

FIG. 3 shows cell staining results of cells transfected with RU873 and RU879 with anti-FLAG HRP.

FIGS. 4A and 4B provide a depiction of RUM 873-expressing cells recognized and bound by magnetic beads coated with an anti-FLAG antibody and control cells.

FIG. 5 provides a flow chart of stable cell line generation using RUM as a selectable marker.

FIG. 6 provides the gene map of expression vector p632 with one cassette expressing a target protein flanked by Promoter A and polyA sequence, the other cassette expressing RUM 873 flanked by Promoter B and a polyA sequence.

FIG. 7 shows protein expression levels of various clones expressing target protein #632. The levels of target protein #632 was measured by an ELISA in conditioned media from CHO cells transiently transfected with various vectors. Clones #1-18 are different clones transfected with vectors expressing target protein #632.

FIG. 8 provides the gene map of a dual selection DNA vector (pDual-Selection). There are three gene expression cassettes, RUM cassette, Zeocin resistance cassette, and a third cassette for target gene expression.

FIGS. 9A-9D show staining of RUM positive cells using magnetic beads coated with an anti-FLAG antibody under electronic microscope.

FIG. 10 shows FACS analysis of CHO cells, CHO cells transfected with RUM, and selected RUM positive cells.

FIGS. 11A-11C show percentage of cells expressing target proteins when selected using zeocin only or zeocin and RUM.

### DETAILED DESCRIPTION OF THE INVENTION

The present invention provides a selectable surface marker (also referred to herein as Rapid Universal Selectable Marker, or “RUM”), which enables rapid selection of cells containing a high copy number of a gene of interest and/or high level of gene expression. Unlike traditional methods which rely on cell survival for selection, the methods of the present invention using the RUMs allow cells having high copy number of a gene of interest and/or high level of gene expression be physically separated from the rest of the cells. Gene amplification of a desired level can be achieved through titration of the stringency of the separation means. The methods of the present invention thus avoid use of selective pressure and allow fast and efficient separation of cells of interest from the rest of the cells. The methods are particularly useful for large scale production of gene expression products, such as protein (e.g., antibody) production in commercial settings. Because the growth media for the cells do not need to contain drugs for cell killing or starvation, stable cell lines in the absence of drugs can be established. The present invention thus also provides powerful methods for establishing stable cell lines for gene expression.

In addition to separating cells transfected with a gene of interest from those not transfected, the method of the present application allows for the separation of cells having a high level of gene expression from the rest of the cells, including cells transfected with the same gene of interest yet do not achieve the desired level of gene expression. The effectiveness and specificity of the present method in separating high-expressing cells from low-expressing cells is unexpected.

Moreover, it was surprisingly found that cells expressing surface markers with tag sequences that are less than about 100 amino acids can convey specificity that allows the desired cells be recognized by a separation means. It was further surprisingly found that cells expressing surface markers with tag sequences that are less than about 100 amino acids can be detected and separated by separation means such as magnetic beads coated with ligands that recognize the tag sequence. The interaction between the short amino acids and the ligands on the magnetic beads was surprisingly strong enough to pull cells and magnetic beads together, and such interaction can sustain a relatively stringent washing condition.

Accordingly, the present invention in one aspect provides methods of producing a recombinant cell (such as a recombinant cell having a high copy number of a gene of interest and/or high level of gene expression) using the RUMs.

In another aspect, there is provided a method of large scale production of a gene expression product using the RUMs.

In another aspect, there is provided a method of establishing a stable cell line using the RUMs.

In another aspect, there are provided expression vectors encoding the RUMs and cells comprising the expression vectors. Also provided are gene expression products (such as proteins) and cells obtained using methods described herein. Further provided are kits that are useful for methods described herein.

### DEFINITIONS

“Ligand recognizing the surface marker” refers to a ligand that specifically binds to the surface marker under suitable conditions.

“Supporting material” used herein refers to any compound or material which may provide a physical or chemical means to separating the ligand and the cells bound thereto from the rest of the cells.

The term “vector,” as used herein refers to a nucleic acid which is capable of directing the expression of a gene of interest.

The term “cistron” or “cassette” used interchangeably herein refers to a translational unit comprising the nucleotide sequence coding for a gene of interest or a surface marker adjacent control regions. Two cistrons are positioned “head to tail” in an expression vector when the two cistrons are arranged in tandem, and the expressions proceed in the same direction. Two cistrons are positioned “head to head” in an expression vector when the two promoters of the two cistrons are positioned next to each other on an expression system, with the expressions proceeding in the different directions. Two cistrons are positioned “tail to tail” in an expression vector when the two promoters of the two cistrons are positioned distal from each other on an expression system, with the expressions proceeding in the different directions.

“Secretion signal sequence” or “signal sequence” refers to a nucleic acid sequence encoding for a short signal peptide that can be used to direct a newly synthesized protein of interest through a cellular membrane.

As used herein, the term “bioreactor” refers to a continuous culture device used to grow cells.

Reference to “about” a value or parameter herein includes (and describes) embodiments that are directed to that value or parameter per se. For example, description referring to “about X” includes description of “X.”

It is understood that aspect and embodiments of the invention described herein include “consisting” and/or “consisting essentially of” aspects and embodiments.

### Exemplary Embodiments of the Present Invention

The present invention in some embodiments provide a method of producing a recombinant cell (such as a recombinant cell having a desired copy of a gene of interest and/or a recombinant cell having a desired level of gene expression), comprising: exposing a population of host cells comprising (for example comprising a vector comprising): i) a first nucleic acid sequence comprising a gene of interest, and ii) a second nucleic acid sequence comprising a coding sequence for a surface marker to a separation means that recognizes the surface marker, wherein cells recognized by the separation means can be separated from the rest of the cells.

In some embodiments, the method comprises: a) exposing a population of host cells comprising (for example comprising a vector comprising): i) a first nucleic acid sequence comprising a gene of interest, and ii) a second nucleic acid sequence comprising a coding sequence for a surface marker to a separation means that recognizes the surface marker, and b) separating cells recognized by the separation means from the rest of the cells. In some embodiments, the method comprises a) culturing a population of host cells comprising (for example comprising a vector comprising): i) a first recombinant nucleic acid sequence comprising a gene of interest, and ii) a second recombinant nucleic acid sequence comprising a coding sequence for a surface marker; b) exposing the cultured cells to a separation means that recognizes the surface marker, and c) separating cells recognized by the separation means from the rest of the cells.

In some embodiments, the method comprises a) culturing a population of host cells comprising (for example comprising a vector comprising): i) a first recombinant nucleic acid sequence comprising a gene of interest, and ii) a second recombinant nucleic acid sequence comprising a coding sequence for a surface marker for at least about one, two, three, four, or five days (or at least about one, two, three, four, or five cell doubling cycles); b) exposing the cultured cells to a separation means that recognizes the surface marker, and c) separating cells recognized by the separation means from the rest of the cells.

In some embodiments, the method comprises: a) introducing into a host cell i) a first nucleic acid sequence comprising a gene of interest, and ii) a second nucleic acid sequence comprising a coding sequence for a surface marker; b) culturing the host cell; c) exposing the cultured cells to a separation means that recognizes the surface marker, and d) separating cells recognized by the separation means from the rest of the cells.

In some embodiments, the method comprises: a) introducing into a host cell i) a first nucleic acid sequence comprising a gene of interest, and ii) a second nucleic acid sequence comprising a coding sequence for a surface marker; b) culturing the host cell for at least about one, two, three, four, or five days (or at least about one, two, three, four, or five cell doubling cycles); c) exposing the cultured cells to a separation means that recognizes the surface marker, and d) separating cells recognized by the separation means from the rest of the cells.

In some embodiments, the method comprises: a) exposing a population of host cells comprising (for example comprising a vector comprising): i) a first nucleic acid sequence comprising a gene of interest, and ii) a second nucleic acid sequence comprising a coding sequence for a surface marker to a separation means that recognizes the surface marker, b) separating cells recognized by the separation means from the rest of the cells; and c) washing the cells recognized by the separation means.

In some embodiments, the method comprises: a) exposing a population of host cells comprising (for example comprising a vector comprising): i) a first nucleic acid sequence comprising a gene of interest, and ii) a second nucleic acid sequence comprising a coding sequence for a surface marker to a first separation means that recognizes the surface marker, b) separating cells recognized by the first separation means from the rest of the cells; c) exposing the separated cells to a second separation means that recognizes the surface marker; and d) separating cells recognized by the second separation means from the rest of the cells. In some embodiments, the first separation means and/or the second separation means are ligands specifically recognizing the surface marker. For example, in some embodiments, the method comprises: a) exposing a population of host cells comprising (for example comprising a vector comprising): i) a first nucleic acid sequence comprising a gene of interest, and ii) a second nucleic acid sequence comprising a coding sequence for a surface marker to a first ligand that that specifically recognizes the surface marker, b) separating cells recognized by the first ligand from the rest of the cells; c) exposing the separated cells to a second ligand that specifically recognizes the surface marker; and d) separating cells recognized by the second ligand from the rest of the cells. In some embodiments, one or more steps described above, such as steps a) and b), steps c) and d), or steps a)-d) can be repeated one, two, three, four, five, or more times, with the same or different separation means (such as ligands).

In some embodiments, the method comprises: a) exposing a population of host cells comprising (for example comprising a vector comprising): i) a first nucleic acid sequence comprising a gene of interest, and ii) a second nucleic acid sequence comprising a coding sequence for a surface marker to a ligand that specifically recognizes the surface marker, b) exposing the cells to a blocking agent that competes for the binding to the surface marker; and c) separating cells recognized by the separation means from the rest of the cells. In some embodiments, steps a)-c) are repeated at least one, two, three, four, five, six, seven, eight, nine, ten, or more times. In some embodiments, the concentration of the blocking agent increases at each cycle until the desired properties of the cells are obtained.

In some embodiments, the method comprises: exposing a population of host cells comprising (for example comprising a vector comprising): i) a first nucleic acid sequence comprising a gene of interest, ii) a second nucleic acid sequence comprising a coding sequence for a surface marker; and iii) a third nucleic acid sequence comprising a coding sequence for a selectable marker that is different from the surface marker to a separation means that recognizes the surface marker, wherein cells recognized by the separation means can be separated from the rest of the cells, wherein the method further comprises selecting cells based on the second selectable marker. In some embodiments, the selection based on the second selectable marker is carried out prior to exposing the cells to the separation means. In some embodiments, the selection based on the selectable marker is carried out during exposing the cells to the separation means. In some embodiments, the selection based on the selectable marker is carried out after exposing the cells to the separation means.

In some embodiments, the method comprises: a) culturing a population of host cells comprising (for example comprising a vector comprising): i) a first nucleic acid sequence comprising a gene of interest, ii) a second nucleic acid sequence comprising a coding sequence for a surface marker; and iii) a third nucleic acid sequence comprising a coding sequence for a selectable marker that is different from the surface marker to a separation means that recognizes the surface marker; b) exposing the cultured cells to a separation means that recognizes the surface marker, and c) separating cells recognized by the separation means from the rest of the cells, wherein the method further comprises selecting cells based on the second selectable marker. In some embodiments, the method further comprises introducing the first and second nucleic acid sequences into the cells, for example by transfection. In some embodiments, the method further comprises releasing cells obtained thereby from the separation means. In some embodiments, the method further comprises culturing a cell line obtained thereby under conditions which permit expression of the gene of interest. In some embodiments, the method further comprises harvesting the gene expression product.

In some embodiments, the method comprises: exposing a population of host cells comprising an expression vector comprising: i) a first nucleic acid sequence comprising a gene of interest, ii) a second nucleic acid sequence comprising a coding sequence for a surface marker; and optionally iii) a third nucleic acid sequence comprising a coding sequence for a selectable marker that is different from the surface marker to a separation means that recognizes the surface marker, wherein cells recognized by the separation means can be separated from the rest of the cells, wherein the method further optionally comprises selecting cells based on the second selectable marker. In some embodiments, the method comprises: a) culturing a population of host cells comprising an expression vector comprising: i) a first nucleic acid sequence comprising a gene of interest, ii) a second nucleic acid sequence comprising a coding sequence for a surface marker; and optionally iii) a third nucleic acid sequence comprising a coding sequence for a selectable marker that is different from the surface marker to a separation means that recognizes the surface marker; b) exposing the cultured cells to a separation means that recognizes the surface marker, and c) separating cells recognized by the separation means from the rest of the cells, wherein the method further optionally comprises selecting cells based on the second selectable marker. In some embodiments, the method further comprises introducing the first and second nucleic acid sequences into the cells, for example by transfection. In some embodiments, the method further comprises releasing cells obtained thereby from the separation means. In some embodiments, the method further comprises culturing a cell line obtained thereby under conditions which permit expression of the gene of interest. In some embodiments, the method further comprises harvesting the gene expression product.

In some embodiments, there is provided a method of producing a recombinant cell (such as a recombinant cell having a desired copy of a gene of interest and/or a recombinant cell having a desired level of gene expression), comprising: exposing a population of host cells comprising (for example comprising a vector comprising): i) a first nucleic acid sequence comprising a gene of interest, and ii) a second nucleic acid sequence comprising a coding sequence for a surface marker to a ligand that specifically recognizes the surface marker, wherein cells recognized by the ligand can be separated from the rest of the cells. In some embodiments, the method comprises: exposing a population of host cells comprising (for example comprising a vector comprising): i) a first nucleic acid sequence comprising a gene of interest, and ii) a second nucleic acid sequence comprising a coding sequence for a surface marker to magnetic beads (such as magnetic beads of about 100-200 nm, 200-500 nm, 500 nm-1 micron, 1-5 microns, 5-10 microns, 10-50 microns, 50-100 microns) attached to (such as coated with) a ligand that specifically recognizes the surface marker, wherein cells recognized by the ligand can be separated from the rest of the cells. In some embodiments, the method comprises: exposing a population of host cells comprising (for example comprising a vector comprising): i) a first nucleic acid sequence comprising a gene of interest, and ii) a second nucleic acid sequence comprising a coding sequence for a surface marker to a solid support that specifically recognizes the surface marker, wherein cells recognized by the solid support can be separated from the rest of the cells. In some embodiments, the method comprises: exposing a population of host cells comprising (for example comprising a vector comprising): i) a first nucleic acid sequence comprising a gene of interest, and ii) a second nucleic acid sequence comprising a coding sequence for an antibody, an antibody fragment there of (such as scFv), or a protein recognizing a cellulose membrane to a cellulose membrane, wherein cells recognized by the cellulose membrane can be separated from the rest of the cells. In some embodiments, the method comprises: a) exposing a population of host cells comprising (for example comprising a vector comprising): i) a first nucleic acid sequence comprising a gene of interest, and ii) a second nucleic acid sequence comprising a coding sequence for an antibody or fragment there of (such as scFv) recognizing a cellulose membrane to a cellulose membrane, wherein cells recognized by the cellulose membrane can be separated from the rest of the cells, and b) harvesting gene expression products from the cells. In some embodiments, the steps in the method is carried out continuously.

In some embodiments, there is provided a method of producing a recombinant cell (such as a recombinant cell having a desired copy of a gene of interest and/or a recombinant cell having a desired level of gene expression), comprising: exposing a population of host cells comprising (for example comprising a vector comprising): i) a first nucleic acid sequence comprising a gene of interest, and ii) a second nucleic acid sequence comprising a coding sequence for a surface marker to a separation means (such as a ligand) that recognizes the surface marker, wherein cells recognized by the separation means (such as a ligand) can be separated from the rest of the cells, wherein the surface marker is not a scFv, CD4, MHC class I molecule H-2k^{k}, or LNGFR. In some embodiments, the method comprises: exposing a population of host cells comprising (for example comprising a vector comprising): i) a first nucleic acid sequence comprising a gene of interest, and ii) a second nucleic acid sequence comprising a coding sequence for a surface marker to a separation means (such as a ligand) that recognizes the surface marker, wherein cells recognized by the separation means (such as a ligand) can be separated from the rest of the cells, wherein the surface marker comprises a tag sequence and a transmembrane domain or a membrane anchoring region. In some embodiments, the method comprises: exposing a population of host cells comprising (for example comprising a vector comprising): i) a first nucleic acid sequence comprising a gene of interest, and ii) a second nucleic acid sequence comprising a coding sequence for a surface marker to a separation means (such as a ligand) that recognizes the surface marker, wherein cells recognized by the separation means (such as a ligand) can be separated from the rest of the cells, wherein the surface marker comprises a tag sequence and a transmembrane domain or a membrane anchoring region, wherein the tag sequence is less than about 100 amino acids (including for example less than about any of 90, 80, 70, 60, 50, 40, 30, 20, 10, or 5 amino acids).

In some embodiments, the method comprises: exposing a population of host cells comprising (for example comprising a vector comprising): i) a first nucleic acid sequence comprising a gene of interest, and ii) a second nucleic acid sequence comprising a coding sequence for a surface marker to a separation means (such as a ligand) that recognizes the surface marker, wherein cells recognized by the separation means (such as a ligand) can be separated from the rest of the cells, wherein the surface marker comprises a tag sequence and a transmembrane domain or a membrane anchoring region, wherein the tag sequence is any of the following: FLAG, 2× FLAG, 3× FLAG, 4× FLAG, 5× FLAG, 6× FLAG, or repeat thereof.

In some embodiments, the method comprises: exposing a population of host cells comprising (for example comprising a vector comprising): i) a first nucleic acid sequence comprising a gene of interest, and ii) a second nucleic acid sequence comprising a coding sequence for RUM 873 or RUM 879 to a separation means (such as a ligand) that recognizes the surface marker, wherein cells recognized by the separation means (such as a ligand) can be separated from the rest of the cells. In some embodiments, the method comprises: exposing a population of host cells comprising an expression vector having a same or similar gene map of p632 (or pDual-Selection) to a separation means (such as a ligand) that recognizes the surface marker, wherein cells recognized by the separation means (such as a ligand) can be separated from the rest of the cells.

In some embodiments, there is provided a method of producing a multimeric protein, comprising: a) exposing a population of cells to a separation means, wherein the cells comprise: 1) a first expression vector comprising: i) a first nucleic acid sequence comprising a first gene of interest, and ii) a second nucleic acid sequence comprising a coding sequence for a first surface marker; and 2) a second expression vector comprising: i) a second nucleic acid sequence comprising a second gene of interest, and iv) a fourth nucleic acid sequence comprising a coding sequence for a second surface marker; wherein the first and second surface markers form a heterodimer on the cell surface that is recognizable by the separation means, and wherein the first gene of interest and the second gene of interest produce polypeptides that form the multimeric protein; and b) separating cells recognized by the separation means from the rest of the cells. In some embodiments, there is provided a method of producing an antibody, comprising: a) exposing a population of cells to a separation means, wherein the cells comprise: 1) a first expression vector comprising: i) a first nucleic acid sequence comprising a coding sequence for an antibody heavy chain, and ii) a second nucleic acid sequence comprising a coding sequence for a first surface marker; and 2) a second expression vector comprising: i) a third nucleic acid sequence comprising a coding sequence for an antibody light chain, and iv) a fourth nucleic acid sequence comprising a coding sequence for a second surface marker; wherein the first and second surface markers form a heterodimer on the cell surface that is recognizable by the separation means; and b) separating cells recognized by the separation means from the rest of the cells.

In some embodiments, two separate means are used, one recognizes the first surface marker, and the other recognizes the second surface marker. In such embodiments, the two surface markers may or may not form heterodimers on the cell surface.

The method described herein can be useful, for example, for producing a recombinant cell having a desired copy of a gene of interest, producing a recombinant cell having a desired level of gene expression, separating cells in large scale, gene amplification, gene expression, large scale cell production, large scale protein production, establishing a stable cell line for gene expression, obtaining cells for large scale protein expression, enriching cells having a desired copy of gene of interest, enriching cells having a desired level of gene expression, and monitoring (such as continuously monitoring) gene amplification in a population of host cells. The methods are particularly useful for expressing a gene of interest and production (such as large scale production) of gene expression products, including protein products.

Also provided are cells obtained from methods described herein and methods of using cells obtained by methods described herein. Further provided are gene expression products produced by cells obtained by methods described herein and methods of use thereof.

The present application also provide expression vectors useful for methods described herein. In some embodiments, there is provided an expression vector comprising: i) a first nucleic acid sequence comprising a gene of interest, and ii) a second nucleic acid sequence comprising a coding sequence for a surface marker, wherein the surface marker comprises a tag sequence and a transmembrane domain or a membrane anchoring region, wherein the tag sequence is not scFv, CD4, MHC class I molecule H-2k^{k}, or LNGFR. In some embodiments, there is provided an expression vector comprising: i) a first nucleic acid sequence comprising a gene of interest, and ii) a second nucleic acid sequence comprising a coding sequence for a surface marker, wherein the surface marker comprises a tag sequence and a transmembrane domain or a membrane anchoring region, wherein the tag sequence is less than about 100 amino acids (including for example less than about any of 90, 80, 70, 60, 50, 40, 30, 20, 10, or 5 amino acids). In some embodiments, the tag sequence comprises (or consisting essentially of or consisting of) one or more of the following: FLAG, His, HA, myc, and V5. In some embodiments, the tag sequence is RUM 873 or RUM 879. In some embodiments, the expression vector has a gene map that is the same or similar gene map as p632 (or pDual-Selection). In some embodiments, the expression vector is p632 (or pDual-Selection).

In some embodiments, there is provided an expression vector comprising: i) a first cistron comprising a first promoter and a gene of interest, and ii) a second cistron comprising a second promoter and a coding sequence for a surface marker, wherein the surface marker comprises a tag sequence and a transmembrane domain or a membrane anchoring region. In some embodiments, the first cistron and the second cistron are positioned head to tail on the expression vector. In some embodiments, the first and second cistron are positioned tail to tail on the expression vector. In some embodiments, the first and second cistrons are positioned head to head on the expression vector. In some embodiments, the tag sequence is not scFv, CD4, MHC class I molecule H-2k^{k}, or LNGFR. In some embodiments, the tag sequence is less than about 100 amino acids (including for example less than about any of 90, 80, 70, 60, 50, 40, 30, 20, 10, or 5 amino acids). In some embodiments, the tag sequence comprises (or consisting essentially of or consisting of) one or more of the following: FLAG, His, HA, myc, and V5. In some embodiments, the tag sequence is RUM 873 or RUM 879. In some embodiments, the expression vector has a gene map that is the same or similar gene map as p632 (or pDual-Selection). In some embodiments, the expression vector is p632 (or pDual-Selection).

In some embodiments, there is provided 1) a first expression vector comprising: i) a first nucleic acid sequence comprising a first gene of interest, and ii) a second nucleic acid sequence comprising a coding sequence for a surface marker; and 2) a second expression vector comprising: i) a second nucleic acid sequence comprising a second gene of interest, and iv) a fourth nucleic acid sequence comprising a coding sequence for a surface marker; wherein the first and second surface marker can form a heterodimer on a cell surface that is recognizable by a separation means. In some embodiments, there is provided a cell comprising: 1) a first expression vector comprising: i) a first nucleic acid sequence comprising a first gene of interest, and ii) a second nucleic acid sequence comprising a coding sequence for a surface marker; and 2) a second expression vector comprising: i) a second nucleic acid sequence comprising a second gene of interest, and iv) a fourth nucleic acid sequence comprising a coding sequence for a surface marker; wherein the first and second surface marker form a heterodimer on the cell surface that is recognizable by a separation means.

In some embodiments, there is provided an expression vector comprising i) a first nucleic acid sequence comprising a gene of interest, ii) a second nucleic acid sequence comprising a coding sequence for a surface marker, and iii) a third nucleic acid sequence comprising a coding sequence for a selectable marker that is different from the surface marker, wherein the surface marker comprises a tag sequence and a transmembrane domain or a membrane anchoring region, wherein the tag sequence is not scFv, CD4, MHC class I molecule H-2k^{k}, or LNGFR. In some embodiments, there is provided an expression vector comprising: i) a first nucleic acid sequence comprising a gene of interest, ii) a second nucleic acid sequence comprising a coding sequence for a surface marker, and iii) a third nucleic acid sequence comprising a coding sequence for a selectable marker that is different from the surface marker, wherein the surface marker comprises a tag sequence and a transmembrane domain or a membrane anchoring region, wherein the tag sequence is less than about 100 amino acids (including for example less than about any of 90, 80, 70, 60, 50, 40, 30, 20, 10, or 5 amino acids). In some embodiments, the tag sequence comprises (or consisting essentially of or consisting of) one or more of the following: FLAG, His, HA, myc, and V5.

In some embodiments, there is provided an expression vector comprising: i) a first cistron comprising a first promoter and a gene of interest, ii) a second cistron comprising a second promoter and a coding sequence for a surface marker, and iii) a third cistron comprising a third promoter and a coding sequence for a selectable marker that is different from the surface marker, wherein the surface marker comprises a tag sequence and a transmembrane domain or a membrane anchoring region. In some embodiments, the first cistron and the second cistron are positioned head to tail on the expression vector. In some embodiments, the first and second cistron are positioned tail to tail on the expression vector. In some embodiments, the first and second cistrons are positioned head to head on the expression vector. In some embodiments, the tag sequence is not scFv, CD4, MHC class I molecule H-2k^{k}, or LNGFR. The third cistron can be positioned head to tail, tail to tail, or head to head with the first and/or second cistron, or it can be completely in a separated position on the expression vector. In some embodiments, the tag sequence is less than about 100 amino acids (including for example less than about any of 90, 80, 70, 60, 50, 40, 30, 20, 10, or 5 amino acids). In some embodiments, the tag sequence comprises (or consisting essentially of or consisting of) one or more of the following: FLAG, His, HA, myc, and V5. In some embodiments, the tag sequence is RUM 873 or RUM 879. In some embodiments, the selectable marker is zeocin. In some embodiments, the expression vector has a gene map that is the same or similar gene map as pDual-Selection. In some embodiments, the expression vector is pDual-Selection.

Also provided herein are cells comprising nucleic acids or expression vectors described herein, as well as intermediate compositions generated during the process of carrying out the methods described herein. For example, in some embodiments, there is provided a cell expressing a recombinant surface marker, wherein the surface marker renders the cell sortable through a separation means that recognizes the surface marker. In some embodiments, the surface marker is not scFv, CD4, MHC class I molecule H-2k^{k}, or LNGFR. In some embodiments, the surface marker comprises a tag sequence and a transmembrane domain or a membrane anchoring region, wherein the tag sequence is less than about 100 amino acids (including for example less than about any of 90, 80, 70, 60, 50, 40, 30, 20, 10, or 5 amino acids). In some embodiments, the tag sequence comprises (or consisting essentially of or consisting of) one or more of the following: FLAG, His, HA, myc, and V5.

In some embodiments, there is provided a cell comprising: i) a first nucleic acid sequence comprising a gene of interest, and ii) a second nucleic acid sequence comprising a coding sequence for a surface marker. In some embodiments, the cell comprises an expression vector comprising: i) a first nucleic acid sequence comprising a gene of interest, and ii) a second nucleic acid sequence comprising a coding sequence for a surface marker. In some embodiments, the cell comprises i) a first promoter and a gene of interest, and ii) a second cistron comprising a second promoter and a coding sequence for a surface marker. In some embodiments, therein is provided a composition comprising the cell and a separation means (such as a ligand) recognizing the surface marker on the cell.

In some embodiments, the cell comprises: i) a first nucleic acid sequence comprising a gene of interest, ii) a second nucleic acid sequence comprising a coding sequence for a surface marker, and iii) a third nucleic acid sequence comprising a coding sequence for a selectable marker that is different from the surface marker. In some embodiments, the cell comprises an expression vector comprising i) a first nucleic acid sequence comprising a gene of interest, ii) a second nucleic acid sequence comprising a coding sequence for a surface marker, and iii) a third nucleic acid sequence comprising a coding sequence for a selectable marker that is different from the surface marker. In some embodiments, the cell comprises: i) a first cistron comprising a first promoter and a gene of interest, ii) a second cistron comprising a second promoter and a coding sequence for a surface marker, and iii) a third cistron comprising a third promoter and a coding sequence for a selectable marker that is different from the surface marker. In some embodiments, i) a first nucleic acid sequence comprising a gene of interest, and ii) a second nucleic acid sequence comprising a coding sequence for a surface marker to a first separation means that recognizes the surface marker. In some embodiments, the cell comprises an expression vector comprising: i) a first nucleic acid sequence comprising a gene of interest, and ii) a second nucleic acid sequence comprising a coding sequence for a surface marker to a first separation means that recognizes the surface marker. In some embodiments, the cell comprises i) a first promoter and a gene of interest, and ii) a second cistron comprising a second promoter and a coding sequence for a surface marker. In some embodiments, therein is provided a composition comprising the cell and a separation means (such as a ligand) recognizing the surface marker on the cell.

The cells and vectors described herein are useful for any of the methods described in the present application.

Methods of Producing Cells Having Desired Levels of Gene Amplification and/or Gene Expression

In some embodiments, there is provided a method of producing a recombinant cell having a desired copy of a gene of interest, comprising: exposing a population of host cells comprising (for example comprising a vector comprising): i) a first nucleic acid sequence comprising a gene of interest, and ii) a second nucleic acid sequence comprising a coding sequence for a surface marker to a separation means that recognizes the surface marker, wherein cells recognized by the separation means can be separated from the rest of the cells. In some embodiments, there is provided a method of producing a recombinant cell having a desired copy of a gene of interest, comprising: a) exposing a population of host cells comprising (for example comprising a vector comprising): i) a first nucleic acid sequence comprising a gene of interest, and ii) a second nucleic acid sequence comprising a coding sequence for a surface marker to a separation means that recognizes the surface marker, and b) separating cells recognized by the separation means from the rest of the cells.

In some embodiments, there is provided a method of producing a recombinant cell having a desired level of gene expression, comprising: exposing a population of host cells comprising (for example comprising a vector comprising): i) a first nucleic acid sequence comprising a gene of interest, and ii) a second nucleic acid sequence comprising a coding sequence for a surface marker to a separation means that recognizes the surface marker, wherein cells recognized by the separation means can be separated from the rest of the cells. In some embodiments, there is provided a method of producing a recombinant cell having a desired level of gene expression, comprising: a) exposing a population of host cells comprising (for example comprising a vector comprising): i) a first nucleic acid sequence comprising a gene of interest, and ii) a second nucleic acid sequence comprising a coding sequence for a surface marker to a separation means that recognizes the surface marker, and b) separating cells recognized by the separation means from the rest of the cells.

In some embodiments, there is provided a method of producing a recombinant cell having a desired copy of a gene of interest, comprising: a) culturing a population of host cells comprising (for example comprising a vector comprising): i) a first nucleic acid sequence comprising a gene of interest, and ii) a second nucleic acid sequence comprising a coding sequence for a surface marker; b) exposing the cultured cells to a separation means that recognizes the surface marker, and c) separating cells recognized by the separation means from the rest of the cells. In some embodiments, there is provided a method of producing a recombinant cell having a desired level of gene expression, comprising: a) culturing a population of host cells comprising (for example comprising a vector comprising): i) a first nucleic acid sequence comprising a gene of interest, and ii) a second nucleic acid sequence comprising a coding sequence for a surface marker; b) exposing the cultured cells to a separation means that recognizes the surface marker, and c) separating cells recognized by the separation means from the rest of the cells. In some embodiments, the cells are cultured for at least one, two, three, four, or five days before they are exposed to a separation means. In some embodiments, the cells are cultured for at least one, two, three, four, or five doubling cycles before they are exposed to a separation means.

In some embodiments, the method further comprises introducing the expression vector into the cells, for example by transfection. Thus, for example, in some embodiments, there is provided a method of producing a recombinant cell having a desired copy of a gene of interest, comprising: a) introducing into a host cell i) a first nucleic acid sequence comprising a gene of interest, and ii) a second nucleic acid sequence comprising a coding sequence for a surface marker; b) culturing the host cell; c) exposing the cultured cells to a separation means that recognizes the surface marker, and d) separating cells recognized by the separation means from the rest of the cells. In some embodiments, there is provided a method of producing a recombinant cell having a desired level of gene expression, comprising: a) introducing into a host cell i) a first nucleic acid sequence comprising a gene of interest, and ii) a second nucleic acid sequence comprising a coding sequence for a surface marker; b) culturing the host cell; c) exposing the cultured cells to a separation means that recognizes the surface marker, and d) separating cells recognized by the separation means from the rest of the cells. In some embodiments, the cells are cultured for at least one, two, three, four, or five days post transfection before they are exposed to a separation means. In some embodiments, the cells are cultured for at least one, two, three, four, or five doubling cycles post transfection before they are exposed to a separation means.

In some embodiments, the method further comprises washing the cells bound to the separation means. Thus, for example, in some embodiments, there is provided a method of producing a recombinant cell having a desired copy of a gene of interest, comprising: a) exposing a population of host cells comprising (such as comprising a vector comprising): i) a first nucleic acid sequence comprising a gene of interest, and ii) a second nucleic acid sequence comprising a coding sequence for a surface marker to a separation means that recognizes the surface marker, b) separating cells recognized by the separation means from the rest of the cells; and c) washing the cells recognized by the separation means. In some embodiments, there is provided a method of producing a recombinant cell having a desired level of gene expression, comprising: comprising: a) exposing a population of host cells comprising (such as comprising a vector comprising): i) a first nucleic acid sequence comprising a gene of interest, and ii) a second nucleic acid sequence comprising a coding sequence for a surface marker to a separation means that recognizes the surface marker, b) separating cells recognized by the separation means from the rest of the cells; and c) washing the cells recognized by the separation means. In some embodiments, the washing solution comprising blocking agents that compete with the cells for binding to the separation means. By controlling the concentration of the blocking agent, cells expressing high level of the surface marker can be selectively obtained.

The desired copy of the genes or a desired level of expression depends on the context. For example, in some embodiments, a cell is deemed to have a desired copy of the genes or a desired level if gene expression if the cell contains at least one copy of the gene of interest. In other words, cells containing the nucleic acid sequence comprising a coding sequence for a surface marker and the nucleic acid sequence comprising the gene of interest can be separated from those not containing these nucleic acid sequences.

In some embodiments, the desired copy of the genes or a desired level of expression is a relatively high copy number or high expression comparing with the rest of the cells in the cell population. This can be achieved, for example, by separating cells expressing high levels of surface markers from those expressing no or low levels of surface markers. The stringency of the separation condition can be controlled in such a way that cells expressing a high level of surface markers can be separated from the rest of the cells. The methods can be carried out repeatedly, for example under the same conditions or under increasing stringent separation conditions, until a desired cell population is obtained. Thus, some embodiments, there is provided a method of isolating cells having a desired level of expression of a gene of interest from a population of cells comprising (such as comprising a vector comprising): i) a first nucleic acid sequence comprising a gene of interest, and ii) a second nucleic acid sequence comprising a coding sequence for a surface marker to a separation means that recognizes the surface marker, wherein the method comprises exposing the population of the cells to a separation means that recognizes the surface marker. In some embodiments, the method further comprises separating cells recognized by the separation means from the rest of the cells. In some embodiments, the method further comprises washing the cells recognized by the separation means.

In some embodiments, the stringency of the separation condition is controlled by controlling the strength of the separation means (such as the binding affinity of a ligand). Thus, for example, in some embodiments, there is provided a method of producing a recombinant cell having a desired copy of a gene of interest (or recombinant cells having high level of gene expression), comprising: a) exposing a population of host cells comprising (such as comprising a vector comprising): i) a first nucleic acid sequence comprising a gene of interest, and ii) a second nucleic acid sequence comprising a coding sequence for a surface marker to a first separation means that recognizes the surface marker, b) separating cells recognized by the first separation means from the rest of the cells; c) exposing the separated cells to a second separation means that recognizes the surface marker; and d) separating cells recognized by the second separation means from the rest of the cells. In some embodiments, the first separation means and/or the second separation means are ligands specifically recognizing the surface marker. For example, in some embodiments, there is provided a method of producing a recombinant cell having a desired copy of a gene of interest (or recombinant cells having high levels of gene expression), comprising: a) exposing a population of host cells comprising (such as comprising a vector comprising): i) a first nucleic acid sequence comprising a gene of interest, and ii) a second nucleic acid sequence comprising a coding sequence for a surface marker to a first ligand that that specifically recognizes the surface marker, b) separating cells recognized by the first ligand from the rest of the cells; c) exposing the separated cells to a second ligand that specifically recognizes the surface marker; and d) separating cells recognized by the second ligand from the rest of the cells. In some embodiments, one or more steps described above, such as steps a) and b), steps c) and d), or steps a)-d) can be repeated one, two, three, four, five, or more times, with the same or different separation means (such as ligands).

Alternatively, when a washing step is used in the method, the stringency of the separation condition can be controlled by the washing conditions. Thus, in some embodiments, the methods described above further may comprise washing cells exposed to a separation means prior to separating the cells from the rest of the cells. In some embodiments, the washing is carried out by using washing solutions. In some embodiments, washing solutions having different stringencies are introduced to the cells exposed to the separation means. In some embodiments, the washing includes introducing blocking agent (such as competing ligands recognizable by the separation means) to the cells exposed to the separation means. In some embodiments, different concentrations of the blocking agents are introduced to the cells exposed to the separation means. By controlling the stringency of the binding condition and/or concentration of the blocking agents introduced to the cells, cells having a desired property can be obtained. Thus, for example, in some embodiments, there is provided a method of producing a recombinant cell having a desired copy of a gene of interest (or recombinant cells having a desired level of gene expression), comprising: a) exposing a population of host cells comprising (for example comprising a vector comprising): i) a first nucleic acid sequence comprising a gene of interest, and ii) a second nucleic acid sequence comprising a coding sequence for a surface marker to a separation means that recognizes the surface marker, b) washing the cells exposed to the separation means; and c) separating cells recognized by the separation means from the rest of the cells. In some embodiments, one or more steps described above (such as steps a) through c), steps b) though c), etc.) are repeated at least one, two, three, four, five, six, seven, eight, nine, ten, or more times. In some embodiments, the steps of the method are carried out continuously. The methods may be carried out with or without changing the conditions in between the steps. Changing conditions may include, for example, varying culture conditions such as media components or physical-chemical parameters. In some embodiments, different separation means are used in different steps.

In some embodiments, there is provided a method of producing a recombinant cell having a desired copy of a gene of interest (or recombinant cells having a desired level of gene expression), comprising: a) exposing a population of host cells comprising (such as comprising a vector comprising): i) a first nucleic acid sequence comprising a gene of interest, and ii) a second nucleic acid sequence comprising a coding sequence for a surface marker to a ligand that specifically recognizes the surface marker, b) exposing the cells with a blocking agent that competes for the binding to the surface marker; and c) separating cells recognized by the separation means from the rest of the cells. In some embodiments, steps a)-c) are repeated at least one, two, three, four, five, six, seven, eight, nine, ten, or more times. In some embodiments, the concentration of the blocking agent increases at each cycle until a desired property of the cells are obtained. In some embodiments, there is provided a method of isolating a recombinant cell having a desired copy of a gene of interest (or recombinant cells having a desired level of gene expression) from a population of cells comprising (such as comprising a vector comprising): i) a first nucleic acid sequence comprising a gene of interest, and ii) a second nucleic acid sequence comprising a coding sequence for a surface marker, the method comprising: a) exposing the population of cells to a ligand that specifically recognizes the surface marker, b) exposing the cells with a blocking agent that competes for the binding to the surface marker; and b) separating cells recognized by the separation means from the rest of the cells. In some embodiments, steps a)-c) are repeated at least one, two, three, four, five, six, seven, eight, nine, ten, or more times. In some embodiments, the concentration of the blocking agent increases at each cycle until a desired property of the cells is obtained.

In some embodiments when separation is based on the measurement of the level of surface marker expression on the cells, cells with expression levels that fall within about top 50%, for example about any of top 40%, 30%, 20%, 10%, 5%, 1%, 0.5%, 0.1%, 0.01%, 0.001%, 0.0001%, or higher can be selected based on such measurement. For example, in some embodiments, there is provided a method of producing a recombinant cell having a desired copy of a gene of interest (or recombinant cells having a desired level of gene expression), comprising: a) measuring the expression level of a surface marker in a population of host cells comprising (for example comprising a vector comprising): i) a first nucleic acid sequence comprising a gene of interest, and ii) a second nucleic acid sequence comprising a coding sequence for a surface marker to a ligand that specifically recognizes the surface marker, b) selecting cells with expression levels that fall within the about top 50%, for example about any of top 40%, 30%, 20%, 10%, 5%, 1%, 0.5%, 0.1%, 0.01%, 0.001%, 0.0001%.

Cells bound to the separation means can be further cultured in the presence of the separation means before getting harvested. For example, when magnetic beads coated with a ligand for the surface marker are used in the methods, the cells can be cultured in the presence of the magnetic beads. The magnetic beads will eventually be diluted out and cleaned away from the cells.

In some embodiments, the cells are first released from the separation means before they are cultured and harvested. For example, when magnetic beads coated with a ligand for the surface marker are used in the methods, the magnetic beads can first be removed from the cells before the cells are further cultured. In some embodiments, the binding between the ligand and the surface marker is condition-specific. In this case, the cells can be released from the separation means by altering the condition. For example, in some embodiments, a calcium-dependent anti-FLAG antibody is used as a ligand for the separation of cells expressing FLAG-containing surface markers. In such embodiments, the cells can be released by reducing the concentration of calcium in the medium, for example by adding a chelating agent (such as EDTA) to the medium.

The methods described herein can further comprise harvesting the cells produced. In some embodiments, the method further comprises expressing the gene expression product from the cells. In some embodiments, the method further comprises isolating gene expression products produced in the cells.

The gene copy number and gene expression level of the gene of interest in the cells can further be measured, for example by ELISA, FACS, Northern blot, or RT-PCR. Cells exhibiting high levels of the gene of interest can then further be selected based on such measurement.

Also provided herein are methods of monitoring gene amplification in a population of cells. This can be useful, for example, in monitoring a cell culture for gene expression, assessing stability of a cell line, determining whether a cell culture remains stable, and determining whether a cell culture remains suitable for continued use. For example, in some embodiments, there is provided a method of monitoring gene amplification in a population of host cells, comprising: a) exposing a population of cells comprising (for example comprising a vector comprising): i) a first nucleic acid sequence comprising a gene of interest, and ii) a second nucleic acid sequence comprising a coding sequence for a surface marker to a separation means that recognizes the surface marker, wherein cells recognized by the separation means can be separated from the rest of the cells. In some embodiments, there is provided a method of determining whether a cell culture is suitable for use in gene expression, wherein cells in the cell culture comprises: i) a first nucleic acid sequence comprising a gene of interest, and ii) a second nucleic acid sequence comprising a coding sequence for a surface marker to a separation means that recognizes the surface marker, the method comprising: 1) obtaining a sample of cells from the cell culture; and 2) exposing to the cell sample a separation means that recognizes the surface marker, wherein the cell culture is suitable for gene expression if at least about 50%, 60%, 70%, 70, 80%, 90%, or 100% cells in the cell sample are recognized by the separation means. In some embodiments, FACS is used to determine the percentage of cells that are recognized by the separation means. In some embodiments, magnetic beads are used to determine the percentage of cells that are recognized by the separation means. The methods can be carried out as frequently as needed during the process of cell culturing and are useful for monitoring cell cultures for gene expression.

In some embodiments, there is provided a method of determining whether a cell culture is unsuitable for use in gene expression, wherein cells in the cell culture comprises: i) a first nucleic acid sequence comprising a gene of interest, and ii) a second nucleic acid sequence comprising a coding sequence for a surface marker to a separation means that recognizes the surface marker, the method comprising: 1) obtaining a sample of cells from the cell culture; and 2) exposing to the cell sample a separation means that recognizes the surface marker, wherein the cell culture is unsuitable for gene expression if less than about any of 50%, 60%, 70%, 70, 80%, 90%, or 100% cells in the cell sample are recognized by the separation means. Cells determined to be unsuitable for gene expression can be treated, such as using methods described herein, to re-enrich the cell population with cells having high gene copy number and high levels of gene expression.

Although the methods in this section focuses on methods of producing a recombinant cell having a desired copy of a gene of interest or a desired level of expression, it is to be understood that the methods are also useful for large scale production of gene expression products, establishment of stable cell lines, enriching cells having high copy of gene of interest or high level of gene expression, and other uses described herein. Some of these uses are described below in more detail.

### Methods of Large Scale Production of Gene Expression Products

The methods described herein can be useful for large scale protein production. In some embodiments, there is provided a method of large scale protein production, comprising: a) culturing a population of host cells comprising (for example comprising a vector comprising): i) a first nucleic acid sequence comprising a gene of interest, wherein the gene of interest codes the protein, and ii) a second nucleic acid sequence comprising a coding sequence for a surface marker; b) exposing the cultured cells to a separation means that recognizes the surface marker, and c) separating cells recognized by the separation means from the rest of the cells. In some embodiments, there is provided a method of large scale protein production, comprising: a) culturing a population of host cells comprising a vector comprising: i) a first nucleic acid sequence comprising a gene of interest, wherein the gene of interest codes the protein, and ii) a second nucleic acid sequence comprising a coding sequence for a surface marker; b) exposing the cultured cells to a separation means that recognizes the surface marker, and c) separating cells recognized by the separation means from the rest of the cells. In some embodiments, the method further comprises introducing the expression vector into the cells, for example by transfection. In some embodiments, the method further comprises harvesting the cells produced. In some embodiments, the method further comprises expressing the protein in the cells. In some embodiments, the method further comprises harvesting the proteins produced in the cells. In some embodiments, the cell culturing is carried out in a bioreactor. In some embodiments, the method is for production of proteins at gram scale. In some embodiments, the method is for production of protein at kilogram scale. In some embodiments, the method is for production of protein at the scale of 1, 10, 100, 1000, 10,000, or 100,000 grams. In some embodiments, the initial cell population is at least about any of 10×10⁴, 10×10⁵, 10×10⁶, 10×10⁷, or 10×10⁸ cells. In some embodiments, the culturing is carried out in a bioreactor, such as a perfusion bioreactor.

Also provided are methods of growing cells for large scale protein production. In some embodiments, there is provided a method of growing cells in large scale, comprising a) culturing a population of host cells comprising (for example comprising a vector comprising): i) a first nucleic acid sequence comprising a gene of interest, and ii) a second nucleic acid sequence comprising a coding sequence for a surface marker on a solid support specifically recognizing the surface marker; and 2) washing the solid support. Cells not expressing high levels of surface markers can be washed off, while cells expressing the surface marker will stay. In some embodiments, the method further comprises harvesting the cells. In some embodiments, the method further comprises harvesting gene expression products (such as proteins) from the cells. For example, in some embodiments, the gene expression product is secreted into the growth medium. The harvesting thus can be achieved by collecting the cell growth medium and isolating the gene expression product from the growth medium. This process can be carried out continuously, and is particularly suitable for large scale protein production in a commercial setting.

Also provided are methods of monitoring a cell culture for large scale protein production. For example, in some embodiments, there is provided a method of monitoring a cell culture for large scale protein production, wherein the cells comprise: i) a first nucleic acid sequence comprising a gene of interest, and ii) a second nucleic acid sequence comprising a coding sequence for a surface marker, comprising: 1) obtaining a sample of cells from the cell culture; and 2) exposing the sample of cells to a separation means that recognizes the surface marker. In some embodiments, there is provided a method of determining whether a cell culture is suitable for use in large scale protein production, wherein cells in the cell culture comprises: i) a first nucleic acid sequence comprising a gene of interest, and ii) a second nucleic acid sequence comprising a coding sequence for a surface marker to a separation means that recognizes the surface marker, the method comprising: 1) obtaining a sample of cells from the cell culture; and 2) exposing to the cell sample a separation means that recognizes the surface marker, wherein the cell culture is suitable for large scale protein production if at least about 50%, 60%, 70%, 70, 80%, 90%, or 100% cells in the cell sample are recognized by the separation means. In some embodiments, FACS is used to determine the percentage of cells that are recognized by the separation means. In some embodiments, magnetic beads are used to determine the percentage of cells that are recognized by the separation means. The methods can be carried out as frequently as needed during the process of cell culturing and are useful for monitoring cell cultures for large scale protein production

In some embodiments, there is provided a method of determining whether a cell culture is unsuitable for use in large scale protein production, wherein cells in the cell culture comprises: i) a first nucleic acid sequence comprising a gene of interest, and ii) a second nucleic acid sequence comprising a coding sequence for a surface marker to a separation means that recognizes the surface marker, the method comprising: 1) obtaining a sample of cells from the cell culture; and 2) exposing to the cell sample a separation means that recognizes the surface marker, wherein the cell culture is unsuitable for large scale protein production if less than about any of 50%, 60%, 70%, 70, 80%, 90%, or 100% cells in the cell sample are recognized by the separation means. Cells determined to be unsuitable for gene expression can be treated, such as using methods described herein, to re-enrich the cell population with cells having high gene copy number and high levels of gene expression.

Thus, in some embodiments, there is provided a method of enriching a population cells for high level of protein expression, wherein the cells comprise: i) a first nucleic acid sequence comprising a gene of interest, and ii) a second nucleic acid sequence comprising a coding sequence for a surface marker to a separation means that recognizes the surface marker, the method comprising: exposing the cells to a separation means that recognizes the surface marker, wherein cells recognized by the separation means can be separated from the rest of the cells. In some embodiments, the method further comprises separating cells recognized by the separation means from the rest of the cells.

### Methods of Establishing Stable Cell Lines

Also provided herein are methods of establishing stable cell lines for gene expression. In some embodiments, there is provided a method of establishing a stable cell line for gene expression, comprising: exposing a population of host cells comprising (for example comprising a vector comprising): i) a first nucleic acid sequence comprising a gene of interest, and ii) a second nucleic acid sequence comprising a coding sequence for a surface marker to a separation means that recognizes the surface marker, wherein cells recognized by the separation means can be separated from the rest of the cells. In some embodiments, there is provided a method of establishing a stable cell line for gene expression, comprising: a) exposing a population of host cells comprising (for example comprising a vector comprising): i) a first nucleic acid sequence comprising a gene of interest, and ii) a second nucleic acid sequence comprising a coding sequence for a surface marker to a separation means that recognizes the surface marker, and b) separating cells recognized by the separation means from the rest of the cells. In some embodiments, there is provided a method of establishing a stable cell line for gene expression, comprising: a) culturing a population of host cells comprising (for example comprising a vector comprising): i) a first nucleic acid sequence comprising a gene of interest, and ii) a second nucleic acid sequence comprising a coding sequence for a surface marker; b) exposing the cultured cells to a separation means that recognizes the surface marker, and c) separating cells recognized by the separation means from the rest of the cells. In some embodiments, the method further comprises introducing the first and second nucleic acid sequences into the cells, for example by transfection. In some embodiments, the method further comprises releasing cells obtained thereby from the separation means. In some embodiments, the method further comprises culturing a cell line obtained thereby under conditions which permit expression of the gene of interest. In some embodiments, the method further comprises harvesting the gene expression product.

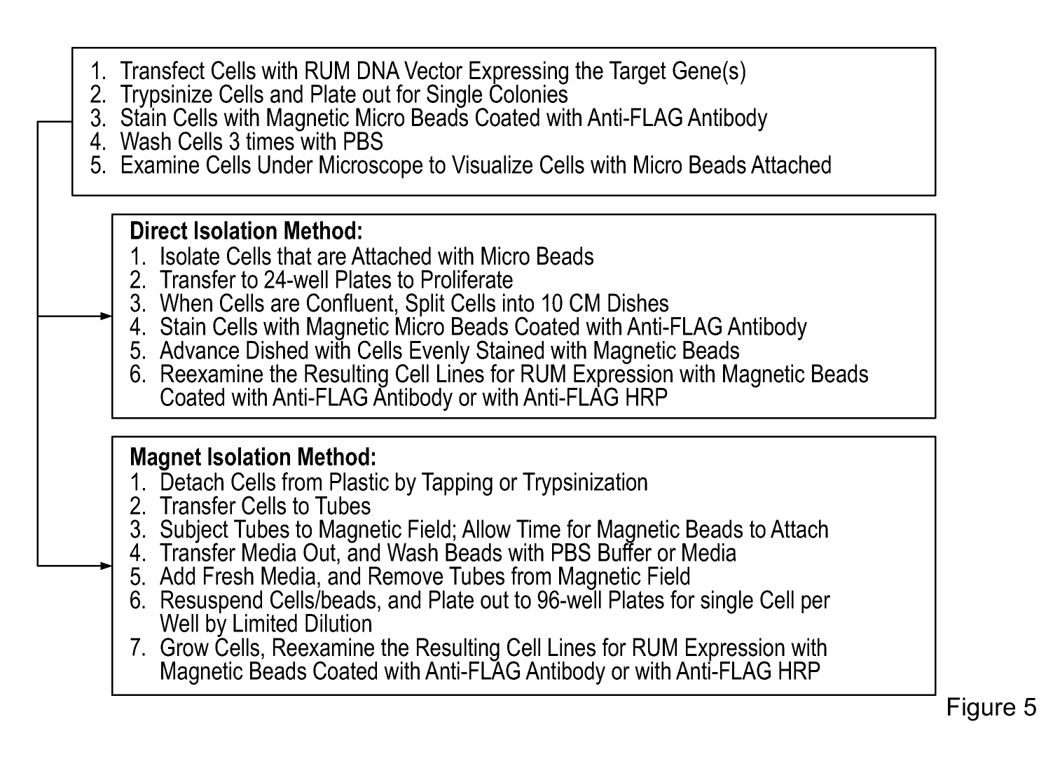

A flow chart of exemplary methods of establishing stable cell lines is provided in FIG. 5. In some embodiments, the method is a direct isolation method as depicted in FIG. 5. In some embodiments, the method is a magnetic method depicted in FIG. 5. It is understood that variations of the methods depicted in FIG. 5 are also contemplated.

In some embodiments, the cells isolated exhibit stable expression of the gene of interest in the absence of any drug selection for at least 10, 20, 30, 40, 50, 60 or more doubling cycles. In some embodiments, the cells selected does not lose more than about 20%, 15%, 10%, 5%, or 1% of its specific productivity after 15 doubling cycles. In some embodiments, the cells do not lose more than about 20%, 15%, 10%, 5%, or 1% of its specific productivity after 50 doubling cycles.

The methods provided herein can also be useful for re-enriching scaled-up stable cell lines in case of heterogeneous loss of expression. Thus, for example, in some embodiments, there is provided a method of enriching cells having a desired level of gene expression, comprising exposing a population of host cells grown from an established cell line comprising: i) a first nucleic acid sequence comprising a gene of interest, and ii) a second nucleic acid sequence comprising a coding sequence for a surface marker to a separation means that recognizes the surface marker, wherein cells recognized by the separation means can be separated from the rest of the cells. In some embodiments, there is provided a method of enriching cells having a desired copy of a gene of interest, comprising: a) exposing a population of host cells comprising (for example comprising a vector comprising): i) a first nucleic acid sequence comprising a gene of interest, and ii) a second nucleic acid sequence comprising a coding sequence for a surface marker to a separation means, and b) separating cells recognized by the separation means from the rest of the cells.

In some embodiments, there is provided a method of determining the stability of a cell line comprising: i) a first nucleic acid sequence comprising a gene of interest, and ii) a second nucleic acid sequence comprising a coding sequence for a surface marker to a separation means that recognizes the surface marker, the method comprising: 1) culturing cells from the cell line, 2) exposing the cells to a separation means that recognizes the surface marker, wherein the cell dine is stable if about any of 50%, 60%, 70%, 70, 80%, 90%, or 100% cells in the cell sample are recognized by the separation means. In some embodiments, the cells are cultured for at least 2, 3, 4, 5, 6, 7, 8, 9, 10, 15, or 20 doubling cycles prior to being exposed to a separation means. In some embodiments, the cells are cultured for at least 2, 3, 4, 5, 6, 7, 8, 9, 10, 15, or 20 passages prior to being exposed to a separation means. In some embodiments, the exposure step is carried out at multiple time points during the culturing process in order to determine the stability of the cell line (e.g., stable after 5 cycles or passages, etc.).

### Double Selection Methods

The methods described herein can further include a second selectable marker, which can be a different surface marker or a different type of selectable marker. The inclusion of a second selectable marker increases selectability and allows more efficient selection. This double selection method is especially suitable for the establishment of a stable cell line having high expression level of the gene of interest. The double selection method is particularly useful for the establishment of stable cell lines for difficult-to-clone genes, such as genes having a large size, genes that cause genomic instability, and/or genes that produce gene expression products that negatively impacts cell growth. By flanking the target gene with two selectable markers and selecting cells based on both selectable markers, the likelihood of a cell harboring the entire target gene is significantly increased.

Thus, for example, in some embodiments, there is provided a method of establishing a stable cell line, comprising: exposing a population of host cells comprising (for example comprising a vector comprising): i) a first nucleic acid sequence comprising a gene of interest, ii) a second nucleic acid sequence comprising a coding sequence for a surface marker; and iii) a third nucleic acid sequence comprising a coding sequence for a selectable marker that is different from the surface marker to a separation means that recognizes the surface marker, wherein cells recognized by the separation means can be separated from the rest of the cells, wherein the method further comprises selecting cells based on the second selectable marker. In some embodiments, there is provided a method of establishing a stable cell line for gene expression, comprising: a) exposing a population of host cells comprising (for example comprising a vector comprising): i) a first nucleic acid sequence comprising a gene of interest, ii) a second nucleic acid sequence comprising a coding sequence for a surface marker; and iii) a third nucleic acid sequence comprising a coding sequence for a selectable marker that is different from the surface marker to a separation means that recognizes the surface marker, and b) separating cells recognized by the separation means from the rest of the cells, wherein the method further comprise selecting cells based on the second selectable marker. In some embodiments, there is provided a method of establishing a stable cell line for gene expression, comprising: a) culturing a population of host cells comprising (for example comprising a vector comprising): i) a first nucleic acid sequence comprising a gene of interest, ii) a second nucleic acid sequence comprising a coding sequence for a surface marker; and iii) a third nucleic acid sequence comprising a coding sequence for a selectable marker that is different from the surface marker to a separation means that recognizes the surface marker; b) exposing the cultured cells to a separation means that recognizes the surface marker, and c) separating cells recognized by the separation means from the rest of the cells, wherein the method further comprises selecting cells based on the second selectable marker. In some embodiments, the method further comprises introducing the first, second, and third nucleic acid sequences into the cells, for example by transfection. In some embodiments, the method further comprises releasing cells obtained thereby from the separation means. In some embodiments, the method further comprises culturing a cell line obtained thereby under conditions which permit expression of the gene of interest. In some embodiments, the method further comprises harvesting the gene expression product.

### Surface Marker

The methods described herein use a surface marker (“RUM”) as a selectable marker for cells having a desired level of gene copy or gene expression. The surface marker contains a transmembrane domain and/or membrane anchoring region and can thus be positioned on the cell surface. At least a portion of the RUM is on the extracellular part of the cell (designated as the “tag sequence”), rendering it detectable (and/or sortable) by a separation means.

Thus, in some embodiments, the surface marker comprises 1) a tag sequence; and 2) a transmembrane domain. In some embodiments, the surface marker comprises 1) a tag sequence; and 2) a membrane anchoring region. In some embodiments, the surface marker further comprises a linker sequence between the tag sequence and the transmembrane domain or membrane anchoring region.

In some embodiments, the membrane anchoring region is a membrane attachment signal, such as modification site for GPI or myristylization. In some embodiments, the membrane anchoring region is an amino acid sequence on the surface marker that binds to a membrane protein in the cell surface. In some embodiments, the surface marker further comprises a signaling peptide.

The tag sequence on the surface marker in some embodiments can be a peptide that is recognizable by a ligand which is part of the separation means for detecting/recognizing the surface marker-containing cells. Suitable pairs of peptide/ligand include, but are not limited to, antibody/antigen, antigen/antibody, avidin/biotin, biotin/avidin, streptavidin/biotin, biotin/streptavidin, glutathione/GST, GST/glutathione, maltose binding protein/amylose, amylose/maltose binding protein, cellulose binding protein and cellulose, cellulose/cellulose binding protein, etc. In some embodiments, the tag sequence on the surface marker is an epitope for an antibody. In some embodiments, the tag sequence on the surface marker is an antibody or a fragment thereof. In some embodiments, the tag sequence comprises two or more of the same peptide, for example a 3× FLAG and repeats of a 3× FLAG as described herein.

In some embodiments, the tag sequence on the surface marker is less than about 100 amino acids (including for example less than about any of 90, 80, 70, 60, 50, 40, 30, 20, 10, or 5 amino acids). Prior to the present invention, it was generally believed that longer peptides (such as peptides longer than 100 amino acids) expressed on the cell surface are needed to confer specificity and low level of cross-reactivity to the cells for cell sorting purposes. We have found that amino acids shorter than 100 amino acids (such as the 3× FLAG tag sequence shown in Examples described herein) can render the cells sortable. This is surprising in view of the fact that there are numerous cell surface proteins and cell matrix complexes that could prevent interaction between the short amino acid sequences (such as the 3× FLAG epitope) and the corresponding ligand (such as the anti-FLAG antibody).

Furthermore, it was surprisingly found that cells expressing surface markers with tag sequences that are less than 100 amino acids can be detected and separated by magnetic beads, such as magnetic beads of about 1-2 microns. Typical diameters of a cell is about 10-20 microns. It was surprising that the interaction between the short amino acids (such as the 3× FLAG epitopes) and the ligand (such as the anti-FLAG antibody) on the magnetic beads can be strong enough to pull cells and microbeads together, and that such interaction can sustain a relatively stringent washing condition.

In some embodiments, the tag sequence on the surface marker is a FLAG sequence. In some embodiments, the tag sequence on the surface marker is 3× FLAG. In some embodiments, the tag sequence on the surface marker comprises two or more copies of 3× FLAG. In some embodiments, the tag sequence is selected from: his tag or multiple his tags, myc tag or multiple myc tags, HA tag or multiple HA tags, V5 tag or multiple V5 tags, GST tag or multiple GST tags, maltose-binding protein (MBP) tag or multiple MBP tags.

In some embodiments, the surface marker is RUM 873. In some embodiments, the surface marker is RUM 879.

In some embodiments, the tag sequence is resistant to protease generally present in the cell culture medium. In some embodiments, the tag sequence does not get internalized by the cell upon binding of a ligand (such as antibody) to the surface marker. In some embodiments, the nucleic acid sequence encoding the surface marker also comprises a nucleic acid sequence that encodes a signal peptide.

In some embodiments, the surface marker is not expressed endogenously in the host cell. In some embodiments, the surface marker is a protein that is expressed ectopically. Such a surface marker can be derived from a protein that is endogenously expressed in a wild type cell, such as from an intracellular protein or from a protein of the nuclear membrane. In some embodiments, the host cell expresses the surface marker endogenously, but the expression is at a level that is too low to be recognizable by the separation means under suitable conditions.

In some embodiments when multiple gene products are expressed in the cell, for example for the expression of multimeric proteins including antibodies, the two or more genes can be present in the same expression vector or in separate expression vectors. When present in separate expression vectors, two or more different surface markers can be used. These surface markers can be used independently for separating cells as described herein. Alternatively, a selection method can be devised so that only cells expressing both surface markers are recognized by the separation means. For example, the surface markers may form a hetero-oligomer (such as a hetero-dimer) on the cell surface. A ligand that specifically recognizes such hetero-oligomer can then be used to separate cells expressing the surface markers from the rest of the cells. This ensures that only cells expressing all the target genes be selected. The “surface marker” in the context of the methods described herein thus also include heterooligomers formed by separate surface marker units. Exemplary hetero-oligomers include, but are not limited to, insulin receptor.

### Separation Means

Cells expressing a surface marker can be separated from the rest of the cells by a separation means recognizing the surface marker. It is to be understood that the nature of the separation means depends on the nature of the surface marker.

For example, in some embodiments, the separation means is a ligand recognizing the surface marker. In some embodiments, the ligand is an antibody or a fragment thereof recognizing the surface marker on cell surface.

In some embodiments, the ligand is coupled (either directly or indirectly) to a supporting material, which in turn provides a physical or chemical means of separating the cells recognized by the ligand.

In some embodiments, the supporting material is a solid support. For example, the ligand can be coupled, either directly or indirectly, to plates, tubes, bottles, flasks, magnetic beads, magnetic sheets, porous matrices, or any solid surfaces and the like. Agents or molecules that may be used to link the ligand to the solid support include, but are not limited to, lectins, avidin/biotin, inorganic or organic linking molecules. The physical separation can be effected, for example, by filtration, isolation, magnetic field, centrifugation, washing, etc.

In some embodiments, the solid support is a bead, a membrane, a cartridge, a filter, a microtiter plate, a test tube, solid powder, a cast or extrusion molded module, a mesh, a fiber, a magnetic particle composite, or any other solid materials. The solid support may be coated with a substance such as polyethylene, polypropylene, poly(4-methulbutene), polystyrene, polyacrylate, polyethylene terephthalate, rayon, nylon, poly(vinyl butyrate), polyvinylidene difluoride (PCDF), silicones, polyformaldehyde, cellulose, cellulose acetate, nitrocellulose, and the like. In some embodiments, the solid support may be coated with a ligand or impregnated with the ligand.

In some embodiments, the ligand is part of a solid support. For example, in some embodiments, the ligand is cellulose surface that specifically recognizes the surface marker. Some of the examples of surface markers suitable for this purpose include, for example, scFv.

In some embodiments, the supporting material is a magnetic bead. Thus, for example, the cell separation can be achieved by exposing cells to magnetic beads coated with a ligand recognizing the surface marker and applying magnetic field to separate the magnetic beads and cells bound thereto. The elution of the cells can be achieved by using gravity flow, centrifugation, vacuum filtration, or by positive pressure, e.g., using a plunger or air pressure. In some embodiments, the magnetic beads have an average size of about 1-200 microns, such as any of about 1-2 microns, 2-10 microns, 10-30 microns, 30-50 microns, 50-100 microns, and 10-200 microns. In some embodiments, the magnetic beads are monodisperse, or substantially monodisperse. In some embodiments, the magnetic beads are coated, for example with protein A.

One surprising finding of the present invention is that cells with RUM present on the cell surface could be detected with magnetic beads having diameters that are about 1-10 microns. It is surprising, for example, that magnetic beads as small as 1-10 microns can provide strong enough force that pull the cells away from the rest of the cells. This is especially surprising when the surface marker or the tag sequence on the surface marker is a small peptide, such as a peptide that is less than about 100 amino acids.

Other solid support that can be used in the methods described herein include, but are not limited to, gelatin, glass, sepharose macrobeads, dextran microcarriers such as CYTODES® (Pharmacia, Uppsala, Sweden). Also contemplated are polysaccharide such as agrose, alginate, carrageenan, chitin, cellulose, dextran or starch, polyacrylamide, polystyrene, polyacrolein, polyvinyl alcohol, polymethylacrylate, perfluorocarbon, inorganic compounds such as silica, glass, kieselquhr, alumina, iron oxide or other metal oxides, or copolymers consisting of any combination of two or more naturally occurring polymers, synthetic polymers or inorganic compounds.

In some embodiments, cells expressing surface markers can be separated from the rest of the cell population by “panning” with a ligand attached to a solid matrix, e.g., to a plate.

In some embodiments, the support material is soluble. For example, in some embodiments, the ligand is attached to a label which allows cells recognized by the ligand be separable from the rest of the cells. For example, in some embodiments, the ligand is attached to a FTIC label. Cells bound to the labeled ligand can then be separated by methods such as Fluorescence Activated Cell Sorting (FACS). FACS may be used and may have varying degrees of color channels, low angle and obtuse light scattering detecting channels, and impedance channels.

In some embodiments, the ligand is attached to a soluble magnetic tag, wherein the magnetic tag allows the cells expressing the surface marker be separated by magnetic field.

Also contemplated are soluble supporting materials comprising polymers such as dextran, polyethylene glycol, polyvinyl alcohol, or hydrolysed starch which provide affinity-ligand matrix conjugates for use in liquid partitioning.

In some embodiments, the surface marker is an epitope that can be recognized by an antibody or a fragment thereof. For example, antibodies recognizing the surface markers can be directly coupled to magnetic polystyrene particles like Dynal M 450 or similar magnetic particles and used for cell separation. In some embodiments, the antibodies can be biotinylated or conjugated with digoxigenin and used in conjunction with avidin or anti-digoxigenin coated affinity columns. In some embodiments, the antibodies are used in conjunction with colloidal superparamagnetic microparticle having an organic coating by e.g., polysaccharides. These particles can be used having a size of 10 to 200 nm, including for example between 40 and 100 nm, and can either directly conjugated to the antibodies or in combination with anti-immunoglobulin, avidin or anti-hepten-specific microbeads. Polysaccharide-coated superparamagnetic particles are commercially available from Miltenyi Biotec GmbH, Germany.

The cells can be exposed to the separation means in a variety of methods. For example, when the separation means is a ligand specifically recognizing the surface marker, the cells and the ligands can be incubated under suitable binding conditions for about any of 5, 10, 20, 30, 60, or 120 minutes prior to the separation of the cells. If desired, the cell/ligand mixture can then be subject to one or more round of washes with washing solutions.

### Expression Vectors

The present application also provides expression vectors useful for methods described herein.

In some embodiments, the nucleic acid sequence encoding the surface marker and the nucleic acid sequence comprising the gene of interest are located on the same expression vector being transfected into the cell. In some embodiments, the nucleic acid sequence encoding the surface marker and the nucleic acid sequence comprising the gene of interest are located on separate vectors which are co-transfected into the host cells.

When the nucleic acid sequence encoding the surface marker and the nucleic acid sequence comprising the gene of interest are located on the same vector, the vector may comprise at least two promoters, one driving the expression of the nucleic acid encoding the surface marker, and the other one driving the expression of the gene of interest. Alternatively, the nucleic acid encoding the surface marker may be driven by the same promoter as the nucleic acid comprising the gene of interest, and the vector may comprise an internal ribosome entry site (iRES).

In some embodiments, the first nucleic acid further comprises a first promoter operably linked to the nucleic acid sequence comprising the gene of interest. In some embodiments, the second nucleic acid further comprises a second promoter operably linked to the nucleic acid sequence encoding the surface marker. Thus, for example, in some embodiments, there is provided an expression vector comprising: i) a first cistron comprising a first promoter and a gene of interest, and ii) a second cistron comprising a second promoter and a coding sequence for a surface marker, wherein the surface marker comprises a tag sequence and a transmembrane domain or a membrane anchoring region. In some embodiments, the first cistron and the second cistron are positioned head to tail on the expression vector. In some embodiments, the first and second cistron are positioned tail to tail on the expression vector. In some embodiments, the first and second cistrons are positioned head to head on the expression vector. In some embodiments, the tag sequence is not scFv, CD4, MHC class I molecule H-2k^{k}, or LNGFR. In some embodiments, the tag sequence is less than about 100 amino acids (including for example less than about any of 90, 80, 70, 60, 50, 40, 30, 20, 10, or 5 amino acids).

In some embodiments, the first and second promoters are the same. In some embodiments, the first and second promoters are different. In some embodiments, the first promoter is stronger than the second promoter. In some embodiments, the first promoter is weaker than the second promoter.

In some embodiments, there is provided an expression vector comprising i) a first nucleic acid sequence comprising a gene of interest, ii) a second nucleic acid sequence comprising a coding sequence for a surface marker, and iii) a third nucleic acid sequence comprising a coding sequence for a selectable marker that is different from the surface marker, wherein the surface marker comprises a tag sequence and a transmembrane domain or a membrane anchoring region, wherein the tag sequence is not scFv, CD4, MHC class I molecule H-2k^{k}, or LNGFR. In some embodiments, there is provided an expression vector comprising: i) a first nucleic acid sequence comprising a gene of interest, ii) a second nucleic acid sequence comprising a coding sequence for a surface marker, and iii) a third nucleic acid sequence comprising a coding sequence for a selectable marker that is different from the surface marker, wherein the surface marker comprises a tag sequence and a transmembrane domain or a membrane anchoring region, wherein the tag sequence is less than about 100 amino acids (including for example less than about any of 90, 80, 70, 60, 50, 40, 30, 20, 10, or 5 amino acids).

In some embodiments, there is provided an expression vector comprising: i) a first cistron comprising a first promoter and a gene of interest, ii) a second cistron comprising a second promoter and a coding sequence for a surface marker, and iii) a third cistron comprising a third promoter and a coding sequence for a selectable marker that is different from the surface marker, wherein the surface marker comprises a tag sequence and a transmembrane domain or a membrane anchoring region. In some embodiments, the first cistron and the second cistron are positioned head to tail on the expression vector. In some embodiments, the first and second cistron are positioned tail to tail on the expression vector. In some embodiments, the first and second cistrons are positioned head to head on the expression vector. In some embodiments, the tag sequence is not scFv, CD4, MHC class I molecule H-2k^{k}, or LNGFR. The third cistron can be positioned head to tail, tail to tail, or head to head with the first and/or second cistron, or it can be completely in a separated position on the expression vector.

In some embodiments, the tag sequence is less than about 100 amino acids (including for example less than about any of 90, 80, 70, 60, 50, 40, 30, 20, 10, or 5 amino acids).

In some embodiments, there is provided an expression vector comprising i) a first nucleic acid sequence for cloning a gene of interest and ii) a second nucleic acid sequence comprising a coding sequence for a surface marker, wherein the surface marker comprises a tag sequence and a transmembrane domain or a membrane anchoring region, wherein the tag sequence is not scFv, CD4, MHC class I molecule H-2k^{k}, or LNGFR. In some embodiments, there is provided an expression vector comprising i) a first nucleic acid sequence for cloning a gene of interest and, ii) a second nucleic acid sequence comprising a coding sequence for a surface marker, wherein the surface marker comprises a tag sequence and a transmembrane domain or a membrane anchoring region, wherein the tag sequence is less than about 100 amino acids (including for example less than about any of 90, 80, 70, 60, 50, 40, 30, 20, 10, or 5 amino acids).

In some embodiments, there is provided an expression vector comprising i) a first nucleic acid sequence for cloning a gene of interest; ii) a second nucleic acid sequence comprising a coding sequence for a surface marker, and iii) a third nucleic acid sequence comprising a third nucleic acid sequence comprising a coding sequence for a selectable marker that is different from the surface marker. In some embodiments, there is provided an expression vector comprising i) a first nucleic acid sequence for cloning a gene of interest; ii) a second nucleic acid sequence comprising a coding sequence for a surface marker, and iii) a third nucleic acid sequence comprising a third nucleic acid sequence comprising a coding sequence for a selectable marker that is different from the surface marker, wherein the tag sequence is not scFv, CD4, MHC class I molecule H-2k^{k}, or LNGFR. In some embodiments, there is provided an expression vector comprising i) a first nucleic acid sequence for cloning a gene of interest; ii) a second nucleic acid sequence comprising a coding sequence for a surface marker, and iii) a third nucleic acid sequence comprising a third nucleic acid sequence comprising a coding sequence for a selectable marker that is different from the surface marker, wherein the tag sequence is less than about 100 amino acids (including for example less than about any of 90, 80, 70, 60, 50, 40, 30, 20, 10, or 5 amino acids).

In addition to components of vectors which may be required for expression of a nucleic acid sequence, suitable vectors can also include a bacterial origin of replication, multiple cloning sites, and/or mammalian origin of replication (e.g., a SV40 or adenovirus origin of replication).

Thus, for example, the vector used herein may include one or more elements for means of replication, e.g., origin of replication, which can be episomal or chromosomal. In some embodiments, the replication sequence renders the vector capable of both means, such that the vector is capable of self-replication as an extrachromosomal unit and of integration into the chromosome either due to the presence of a translocatable sequence, such as an insertion sequence or transposon, due to substantial homology with a sequence present in the chromosome or due to non-homologous recombinant events. The replication sequence or replicon will be one recognized by the transformed host cell, e.g., an autonomous replicating segment, by itself, or in conjunction with a centromere, or the like. The particular replication sequence is not critical to the subject invention and various sequences can be employed.

In some embodiments, the expression vector is a mammalian expression vector. Suitable vectors include, but are not limited to, those obtainable through a commercial vendor, e.g., Invitrogen (Carlsbad, Calif.), Promega (Madison, Wis.), and Strategene (La Jolla, Calif.), and can be modified as needed. Examples of commercially available vectors include pcDNA3 (Invitrogen); and pCMV-Script (Stratagene).

Suitable promoters useful for the present invention include, but are not limited to, CMV promoter, SV40 promoter, albumin promoter, ubiquitin promoter, beta-actin promoter, elongation factor promoter, and other promoters known in the art.

In some embodiments, the vector comprises at least one promoter of the murine CMV immediate early region. The promoter may for example be the promoter of the mCMV IE1 gene (the “IE1 promoter”). The promoter may also be the promoter of the mCMV IE2 gene (the“IE2 promoter”). In some embodiments, the vector comprises at least two promoters of the murine CMV immediate early region, such as an IE1 promoter and an IE2 promoter.

### Host Cells

Any number of cells may be screened using methods described herein. For example, at least about 10³, 10⁴, 10⁵, 10⁶, 10⁷, 10⁸, 10⁹, 10¹⁰, 10¹¹, 10¹², 10¹³, 10¹⁴, 10¹⁵ cells may be screened. The population of host cells can therefore comprise at least about 10³, 10⁴, 10⁵, 10⁶, 10⁷, 10⁸, 10⁹, 10¹⁰, 10¹¹, 10¹², 10¹³, 10¹⁴, 10¹⁵ cells.

In some embodiments, the cell is a primary cell. In some embodiments, the cell is a hybridoma. In some embodiments, the cell is an established cell line, for example a cell line established from an eukaryote such as plant or animal. In some embodiments, the cell is an eukaryotic cell. In some embodiments, the cell is a yeast cell. In some embodiments, the cell is a mammalian cell, e.g., germ cells and somatic cells derived from mammals, such as mice, rats, or other rodents, or form primates, such as humans or monkeys. Exemplary cells include, for example, Chinese hamster ovary (CHO) (e.g., DG44 and DUXB11), Chinese hamster fibroblast (e.g., R1610), human cervical carcinoma (e.g., HELA), monkey kidney line (e.g., CVI and COS), murine fibroblast (e.g., BALBc/3T3), murine myeloma (NS0; SP2/O), hamster kidney line (e.g., 293 and 293T). In some embodiments, the cell is a CHO-DUKX cell.

In some embodiments, the cells do not express a surface marker (or the tag sequence portion of the surface marker) endogenously. In some embodiments, the cells express the surface marker (or the tag sequence portion of the surface marker) endogenously.

The expression vectors described herein can be introduced into the host cells by various techniques known in the art, such as transformation, transfection, transduction, protoplast fusion, calcium phosphate precipitation, cell fusion with enveloped DNA, microinjection, and infection with intact virus.

The transfection can be effected by any means, physical or biological. Physical means include direct injection, or DEAE-dextran mediated transfection, lipofectamine transfection, electroporation, calcium phosphate mediated or lipid-mediated transfection, and the like.

Methods of culturing cells are known in the art. In some embodiments, the cells can be cultured in a bioreactor according to standard cell culture techniques. Any bioreactor suitable for culturing cells, particularly for producing large quantities of a protein of interest on a commercial scale, can be used in a method described herein, including, for example, fed-batch, perfusion, stirred tank, airlift, and disposable bioreactors. In some embodiments, the bioreactor is a perfusion bioreactor.

### Genes of Interest

The gene of interest discussed herein include nucleic acids (such as cDNA) encoding a polypeptide (such as protein), a peptide, as well as genes encoding nucleic acids.

In some embodiments, the gene of interest encodes a RNA, including for example antisense RNA, siRNA, microRNA, ribozyme, and the like.

In some embodiments, the gene of interest encodes a protein. In some embodiments, the protein is a secreted protein. In some embodiments, the protein is an antibody. In some embodiments, the protein is a membrane protein. In some embodiments, the protein is any one of the following: GPCR, ion channel. In some embodiments, the protein is an intracellular protein, such as an enzyme or a transcription factor.

In some embodiments, the gene expression product (such as protein) is at least about 50 kDa, such as at least about 100, 200, 300, 400, 500, or more kDa. In some embodiments, the expression of the gene expression product (such as protein) negatively impacts (such as slows down) the growth of the host cells.

Other suitable proteins of interest can include any monomeric, dimeric or multimeric proteins. In addition, such proteins can be of the family of CXC chemokines and their receptors, CC chemokines and their receptors, CD proteins, interleukins and their receptors, interferons and their receptors, TNF super family and their receptors, and tumor-associated antigens which may not fall within any of the foregoing families of proteins. Furthermore, the protein of interest can be an antibody, e.g., one that binds to any of the foregoing proteins.

Additional non-limiting examples of proteins of interest include: growth hormones such as human growth hormone, bovine growth hormone, parathyroid hormone, thyroid stimulating hormone, follicle stimulating hormone growth, luteinizing hormone, and hormone releasing factor; lipoproteins; alpha-1-antitrypsin; insulin A-chain; insulin B-chain; proinsulin; calcitonin; glucagon; molecules such as renin; clotting factors such as factor VIIIC, factor IX, tissue factor, and von Willebrands factor; anti-clotting factors such as Protein C, atrial natriuretic factor, lung surfactant; a plasminogen activator, such as urokinase or human urine or tissue-type plasminogen activator (t-PA); bombesin; thrombin; hemopoietic growth factor; tumor necrosis factor-alpha and -beta; enkephalinase; RANTES (regulated on activation normally T-cell expressed and secreted); human macrophage inflammatory protein (MIP-I-alpha); IL-8; chemerin; IP-IO; CCL22; IL-23; SDF-I; IFN alpha; IL33; HIG2; IL-18; a serum albumin such as human serum albumin; mullerian-inhibiting substance; relaxin A- or B-chain; prorelaxin; mouse gonadotropin-associated peptide; IgE; Ig-Kappa; Ig-Lambda; DNase; inhibin; activin; receptors for hormones, growth factors, chemokines and cytokines, such as Neuropilin-1, CXCR4, IFNAR1, IL23R, ChemR23, CCR4, Folate receptor/FLR4, Frizzled-7, Frizzled-10, GITR, CXCR1, CXCR3, IL-1 8R; integrins, adhesion molecules, and their ligands, such as CD1Ia, CD1Ib, CD1Ic, CD18, an ICAM, Beta-4-integrin, VAP-I, VLA-4 and VCAM; protein A or D; rheumatoid factors; a neurotrophic factor such as bone-derived neurotrophic factor (BDNF), neurotrophin-3, -4, -5, or -6 (NT-3, NT-4, NT-5, or NT-6), growth factors including vascular endothelial growth factors (VEGF), nerve growth factor such as NGF-beta; platelet-derived growth factor (PDGF); fibroblast growth factor such as aFGF, bFGF, FGF-4, FGF-5, FGF-6; epidermal growth factor (EGF); transforming growth factor (TGF) such as TGF-alpha and TGF-beta, including TGF-beta-1, TGF-beta-2, TGF-beta-3, TGF-beta-4, or TGF-beta-5; insulin-like growth factor-I and -II (IGF-I and IGF-II); des(1-3)-IGF-I (brain IGF-I), insulin-like growth factor binding proteins; leukocyte surface markers, such as CD3, CD4, CD8, CD19, CD21, CD22, CD25, CD30, CD70, CD200, Lag 3, BTLA, IRTA1, IRTA2, IRTA3, IRTA4, IRTA5, CEACAM1, NKG2D, PD-I; Fc receptors, including CD16, CD32, CD64, CD89 and FcRn; erythropoietin; osteoinductive factors; OSCAR; OSCAR-ligand; a bone morphogenetic protein (BMP); an interferon such as interferon-alpha, -beta, and -gamma; colony stimulating factors (CSFs), e.g., M-CSF, GM-CSF, and G-CSF; interleukins (ILs), e.g., IL-I to IL-1033; superoxide dismutase; T-cell receptors; a cytotoxic T-lymphocyte associated antigen (CTLA), such as CTLA-4; B7 family of proteins, including B7-H1, B7-H3, B7-H4 and B7-DC (PD-L2); tumor associated antigens such as HER2 receptor, HER3 receptor, HER4 receptor, RG-I, hCG, prostate specific membrane antigen (PSMA) receptor, Galectin-1, Galectin-3, Ephrin B3R, Fucosyl GM1, Edge4, Ptk7, Mud, Mesothelin, Glypican 3, MICA, Fibronectin EDB, Testisin, Autotaxin, Hepsin, GPR56, KCNB, GPCR3, IGSF4, KIAA 1455, matriptase, Nucleolin, TMPRSS4, NGEP, PSGR, TF Antigen, RAET1G; decay accelerating factor; viral antigen such as, for example, a portion of the AIDS or SARS envelopes; transport proteins; homing receptors; addressins; regulatory proteins; enzymes; chimeric proteins such as immunoadhesins and fragments of any of the above-listed proteins. Examples of bacterial proteins or proteins include, e.g., Anthrax PA, C. difficile toxins A and B, SLT, alkaline phosphatase and beta-lactamase.

In one embodiment, the protein of interest is an antibody, which can be of any antibody type, e.g., murine, chimeric, humanized and human, or a combination thereof. A DNA sequence encoding an antibody can encode only a fragment of the antibody, e.g., the antigen binding portion or Fc portion, or a combination of both. Those of ordinary skill in the art will appreciate the term “antigen-binding portion” of an antibody refers to one or more fragments of an antibody that retain the ability to bind to an antigen. It has been shown that the antigen-binding function of an antibody can be performed by fragments of a full-length antibody. Examples of binding fragments encompassed within the term “antigen-binding portion” of an antibody include but are not limited to: (i) a Fab fragment, a monovalent fragment consisting of the V_{L}, V_{H}, C_{L} and C_{H} domains; (ii) a F(ab′)2 fragment, a bivalent fragment comprising two Fab fragments linked by a disulfide bridge at the hinge region; (iii) a Fd fragment consisting of the V_{H} and C_{H} domains; (iv) a Fv fragment consisting of the V_{L} and V_{H} domains of a single arm of an antibody, (v) a dAb fragment (Ward, et al., Nature 341:544-546 (1989)), which consists of a V_{H} domain; and (vi) an isolated complementarity determining region (CDR). Furthermore, although the two domains of the Fv fragment, V_{L} and V_{H}, are coded for by separate DNA sequences, they can be joined, using recombinant methods, by a synthetic linker that enables them to be made as a single protein chain in which the V_{L} and V_{H} regions pair to form monovalent molecules (known as single chain Fv (scFv); see e.g., Bird, et al., Science 242:423-426 (1988); and Huston, et al., Proc. Natl. Acad. Sci. USA 85:5879-5883 (1988)). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

### Kits

Also provided herein are kits for carrying out methods described herein. For example, in some embodiments, there is provided a kit comprising an expression vector comprising: a) an expression vector comprising i) a first nucleic acid sequence for cloning a gene of interest and ii) a second nucleic acid sequence comprising a coding sequence for a surface marker, wherein the surface marker comprises a tag sequence and a transmembrane domain or a membrane anchoring region, wherein the tag sequence is not scFv, CD4, MHC class I molecule H-2k^{k}, or LNGFR, and b) an instruction for using the surface marker for a method described herein. In some embodiments, there is provided a kit comprising: a) an expression vector comprising i) a first nucleic acid sequence for cloning a gene of interest and, ii) a second nucleic acid sequence comprising a coding sequence for a surface marker, wherein the surface marker comprises a tag sequence and a transmembrane domain or a membrane anchoring region, wherein the tag sequence is less than about 100 amino acids (including for example less than about any of 90, 80, 70, 60, 50, 40, 30, 20, 10, or 5 amino acids), b) an instruction for using the surface marker for a method described herein. In some embodiments, the kit further comprises a population of cells.

In some embodiments, there is provided a kit comprising a) an expression vector comprising i) a first nucleic acid sequence for cloning a gene of interest; ii) a second nucleic acid sequence comprising a coding sequence for a surface marker, and iii) a third nucleic acid sequence comprising a third nucleic acid sequence comprising a coding sequence for a selectable marker that is different from the surface marker, and b) an instruction for using the surface marker for a method described herein. In some embodiments, there is provided a kit comprising a) an expression vector comprising i) a first nucleic acid sequence for cloning a gene of interest; ii) a second nucleic acid sequence comprising a coding sequence for a surface marker, wherein the tag sequence is not scFv, CD4, MHC class I molecule H-2k^{k}, or LNGFR, and iii) a third nucleic acid sequence comprising a third nucleic acid sequence comprising a coding sequence for a selectable marker that is different from the surface marker, and c) an instruction for using the surface marker for a method described herein. In some embodiments, there is provided a kit comprising: a) an expression vector comprising i) a first nucleic acid sequence for cloning a gene of interest; ii) a second nucleic acid sequence comprising a coding sequence for a surface marker, and iii) a third nucleic acid sequence comprising a third nucleic acid sequence comprising a coding sequence for a selectable marker that is different from the surface marker, wherein the tag sequence is less than about 100 amino acids (including for example less than about any of 90, 80, 70, 60, 50, 40, 30, 20, 10, or 5 amino acids), and b) an instruction for using the surface marker for a method described herein. In some embodiments, the kit further comprises a population of cells.

Those skilled in the art will recognize that several embodiments are possible within the scope and spirit of this invention. The invention will now be described in greater detail by reference to the following non-limiting examples. The following examples further illustrate the invention but, of course, should not be construed as in any way limiting its scope.

### EXAMPLES

### Example 1

### Construction of RUM

RUM protein sequences were designed de novo, and the DNA fragments corresponding to the sequences were produced by gene synthesis using oligonucleotides as building blocks.

RUM 873 was made by a gene synthesis method called Ligase chain reaction using the following 12 oligonucleotides:

100205A
(SEQ ID NO: 1)
ACTGGTGTCCACTCCCTGCAGGGCGGAGGTGGTGCAGGAGGCGGTGGAGACTACAA
100205B
(SEQ ID NO: 2)
AGACCATGACGGTGATTATAAAGATCATGACATCGATTACAAGGACGACGATGACA
100205C
(SEQ ID NO: 3)
AGGGAGGTGGCGGTGGAGGCGGAAATGCTGTGGGCCAGGACACGCAGGAGGTCATC
100205D
(SEQ ID NO: 4)
GTGGTGCCACACTCCTTGCCCTTTAAGGTGGTGGTGATCTCAGCCATCCTGGCCCT
100205E
(SEQ ID NO: 5)
GGTGGTGCTCACCATCATCTCCCTTATCATCCTCATCATGCTTTGGCAGAAGAAGC
100205F
(SEQ ID NO: 6)
CACGTTAGTCTAGAGGGCCCTATTCTAT
100205G
(SEQ ID NO: 7)
ATAGAATAGGGCCCTCTAGACTAACGTGGCTTCTTCTGCCAAAGCATGATGAGGAT
100205H
(SEQ ID NO: 8)
GATAAGGGAGATGATGGTGAGCACCACCAGGGCCAGGATGGCTGAGATCACCACCA
100205I
(SEQ ID NO: 9)
CCTTAAAGGGCAAGGAGTGTGGCACCACGATGACCTCCTGCGTGTCCTGGCCCACA
100205J
(SEQ ID NO: 10)
GCATTTCCGCCTCCACCGCCACCTCCCTTGTCATCGTCGTCCTTGTAATCGATGTC
100205K
(SEQ ID NO: 11)
ATGATCTTTATAATCACCGTCATGGTCTTTGTAGTCTCCACCGCCTCCTGCACCAC
100205L
(SEQ ID NO: 12)
CTCCGCCCTGCAGGGAGTGGACACCAGT

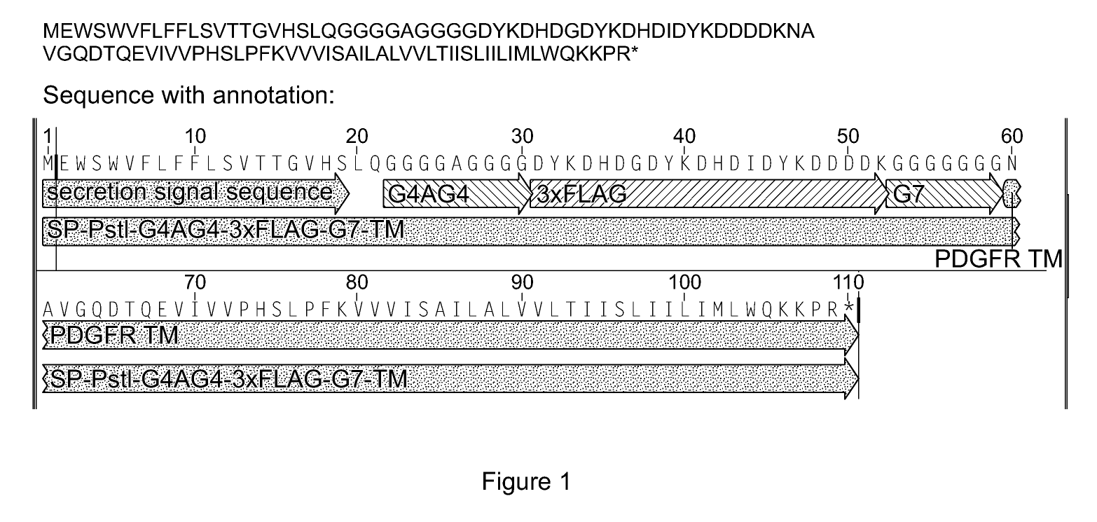

The resulting 308 base pair long RUM 873 was cloned into a DNA vector with HindIII and XbaI restriction enzyme sites. Restriction enzyme digest was used to verify the positive clones. RUM 873 has a secretion signal sequence, followed by a polyglycine linker (G4AG4), a 3× FLAG peptide sequences, a polyglycine linker (G7), and a membrane anchor (PDGFR TM). The protein sequence of RUM 873 is provided below and depicted in FIG. 1:

(SEQ ID NO: 13)
MEWSWVFLFFLSVTTGVHSLQGGGGAGGGGDYKDHDGDYKDHDIDYKDDD
DKNAVGQDTQEVIVVPHSLPFKVVVISAILALVVLTIISLIILIMLWQKK
PR

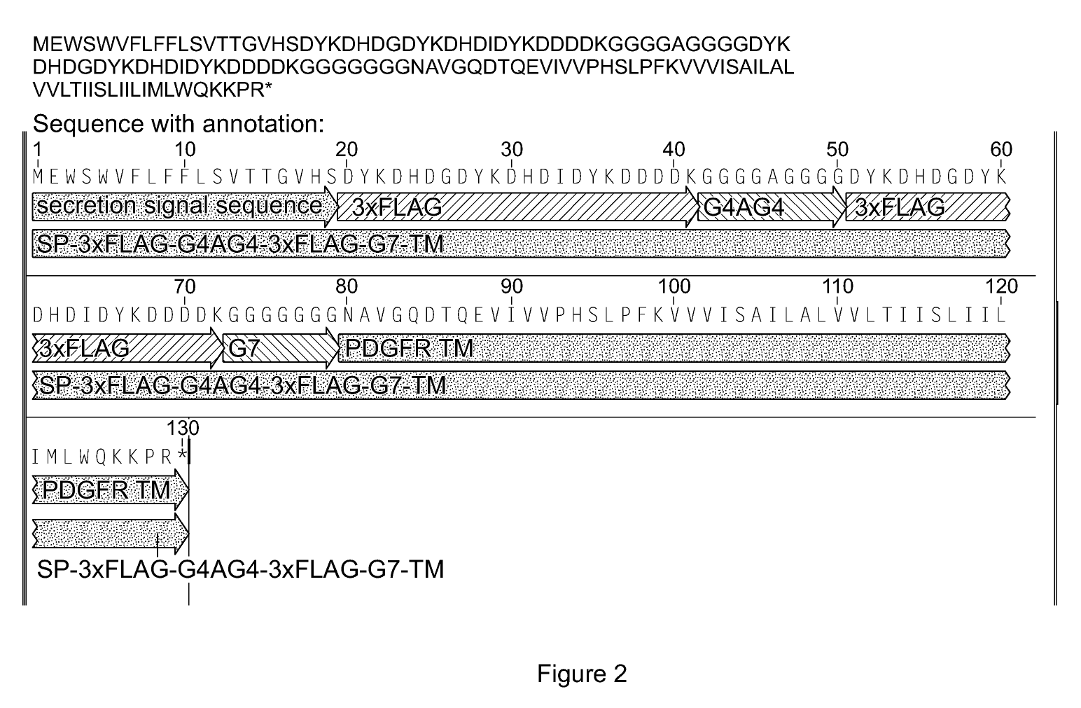

RUM 879 was made by inserting a DNA fragment containing a 3× FLAG sequence into the PstI site of RUM 873 by homologous recombination, which also removed the PstI site. Thus RUM 879 has a secretion signal sequence, followed by a 3× FLAG peptide sequence, a polyglycine linker (G4AG4), a 3× FLAG peptide sequence, a polyglycine linker (G7), and a membrane anchor (PDGFR TM). The protein sequence of RUM 879 is provided below and depicted in FIG. 2:

(SEQ ID NO: 14)
MEWSWVFLFFLSVTTGVHSDYKDHDGDYKDHDIDYKDDDDKGGGGAGGGG
DYKDHDGDYKDHDIDYKDDDDKGGGGGGGNAVGQDTQEVIVVPHSLPFKV
VVISAILALVVLTIISLIILIMLWQKKPR

### Example 2

### Rapid Identification of RUM Positive Cells after Transient Transfection

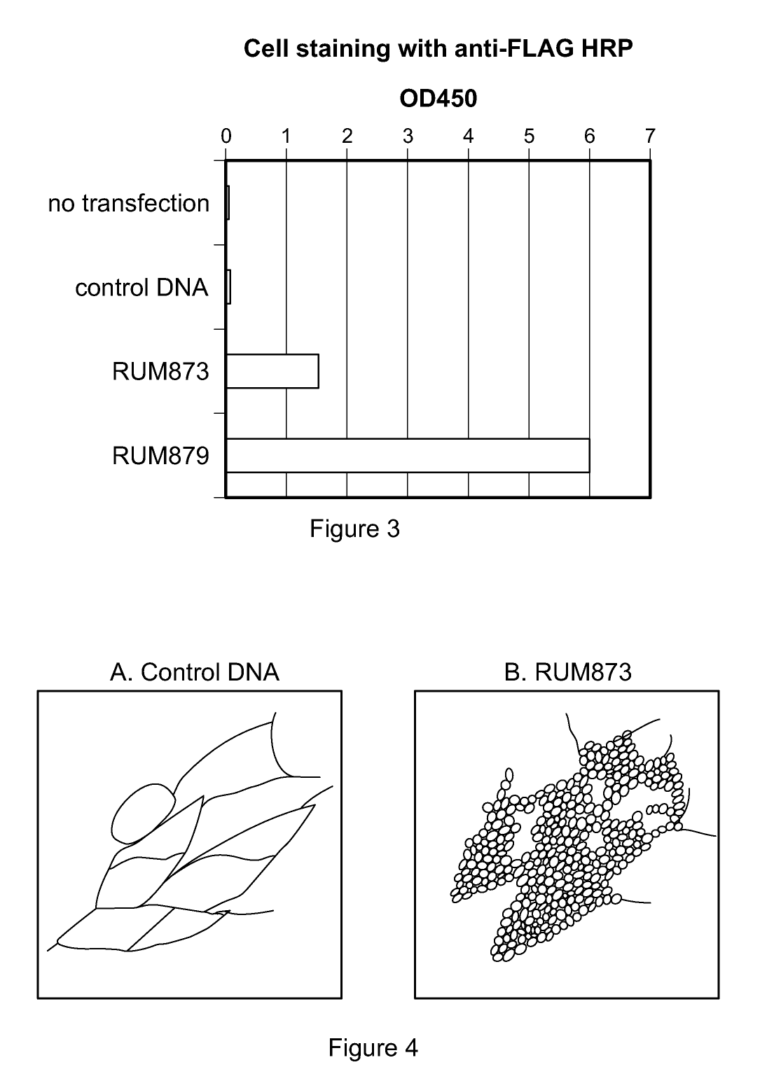

Plasmid DNA of expression vectors containing either RUM 873, RUM 879 or negative controls were individually and transiently transfected into cultured monolayer COST and CHO cells in 24-well tissue culture plates using lipofectamine transfection reagent (Invitrogen). Two days after transfection, these attached cells were incubated for 30 minutes with a FLAG antibody (M2) that has been conjugated with horseradish peroxidase (HRP, Sigma) in the buffer of 500 ul per well of DMEM/F12 with 1% fetal bovine serum. After incubation, the cells were washed twice with 1 ml PBS, and then incubated with 300 ul of a buffer containing TMB (Rockland Immunologicals) which is a substrate for HRP at 37° C. Within 5 minutes, media with cells transfected with RUM 873 or RUM 879 visibly turned blue, while media with cells transfected with control DNA remained colorless. The 100 ul media was transferred to the wells of a 96-well assay plate, and mixed with 100 ul of 1N hydrochloric acid. The addition of hydrochloric acid turned the blue color into yellow. The 96-well plate was then read immediately for absorbance at 450 nm. The FIG. 3 below shows the OD450 absorbance data.

As shown in FIG. 3, cells expressing RUM 873 and RUM 879 were able to produce a HRP signal, while controls cells were not, under the same assay condition. The signal intensity of RUM 879 is significantly higher RUM 873, consistent with the fact that RUM 879 contains two copies of 3× FLAG, while RUM 873 only has one.

### Example 3

### Staining of RUM Positive Cells Using Microbeads

While HRP-based detection method described above were able to easily detect the presence of RUM in culture cells, we sought to staining RUM positive cells using microbeads coated with anti-FLAG antibody.

To experiment with this, we transfected both COS7 and CHO cells with either negative control, RUM 873, or RUM 879 in 24-well plates. Two days after transfection, cells were incubated with 300 ul DMEM/F12 containing 1 ug/ml of anti-FLAG antibody and 0.01 ug/ul of Protein A-coated magnetic beads. After 10 minutes of incubation, the media was removed, and cells were washed twice with PBS by pipetting. When we examined the cells under a microscope, it was observed that very few magnetic beads could be found in wells of cells without RUM, while wells with RUM positive cells had tens of thousands of magnetic beads. Notably, some of cells transfected with RUM were completely coated with magnetic beads, while other cells were not. We speculate that no all cells were expressing RUM effectively—cells with high level of RUM expression could attract many magnetic beads, while cells with low or no levels of RUM could attract few or none.

In a separate experiment, CHO cells expressing either control DNA or RUM 873 were incubated for 30 minutes in media containing magnetic beads (about 1 micron in diameter) coated with anti-FLAG antibodies. The cells were then washed three times with PBS and inspected under microscope. The observation is depicted in FIG. 4. As shown in FIG. 4, cells expressing RUM 873, but not the control cells, were coated with magnetic beads.

It is notable that the interactions appear tight between RUM positive cells and the magnetic beads. Even when we washed the cells vigorously with PBS, there were still plenty of magnetic beads remained on those cells.

### Example 3A

### Staining of RUM Positive Cells Using Magnetic Microbeads

To further examine staining of RUM positive cells using magnetic microbeads, CHO cells were incubated with anti-FLAG antibody and Protein A-coated magnetic beads two days after transfection with RUM 879 DNA. As shown in FIG. 9, some colonies were completely coated with magnetic beads (FIGS. 9A and 9C), while others were not (FIG. 9B). FIG. 9D further depicts two adjacent colonies, with one expressing RUM (upper) and the other not expressing RUM (lower). We speculate that not all cells were expressing RUM effectively—cells with high level of RUM expression could attract many magnetic beads, while cells with low level or no level of RUM could attached few or none.

It is notable that the interactions appear tight between RUM positive cells and the magnetic beads. Even when the cells were vigorously washed with PBS, there were still large numbers of magnetic beads that remained bound to cells.

### Example 4

### Isolation of RUM Positive Cells Using Magnetic Beads and Field

Since magnetic beads could be manipulated with a magnetic field, we sought to isolate magnetic beads-coated mammalian cells using magnets. After treatment of magnetic beads, 100 ul trypsin was added the wells to disrupt cell attachment. After 2 minutes of trypsin treatment, 200 ul of fresh DMEM/F12 media was added to each well, and media was pipetted up and down at least 5 times to facilitate the detachment of mammalian cells. Media containing the cell suspension was then transferred to microtubes, and placed under a magnetic field. Within seconds, magnetic beads moved to the side of microtubes closest to the magnetic field, and accumulated there. With the tubes firmly under the magnetic field, media was pipetted out, and cells were washed twice with fresh DMEM/F12. After the wash, microtubes were removed from the magnet, and resuspended in media before transferred to new tissue culture plates. These plates were incubated in mammalian cell incubator. After 5 days of incubation, colonies were obvious under the microscope. and presence of RUM could be detected with HRP-based method.

### Example 5

### Generation of Stable Cells Expressing a Target Protein Using RUM as Selectable Marker

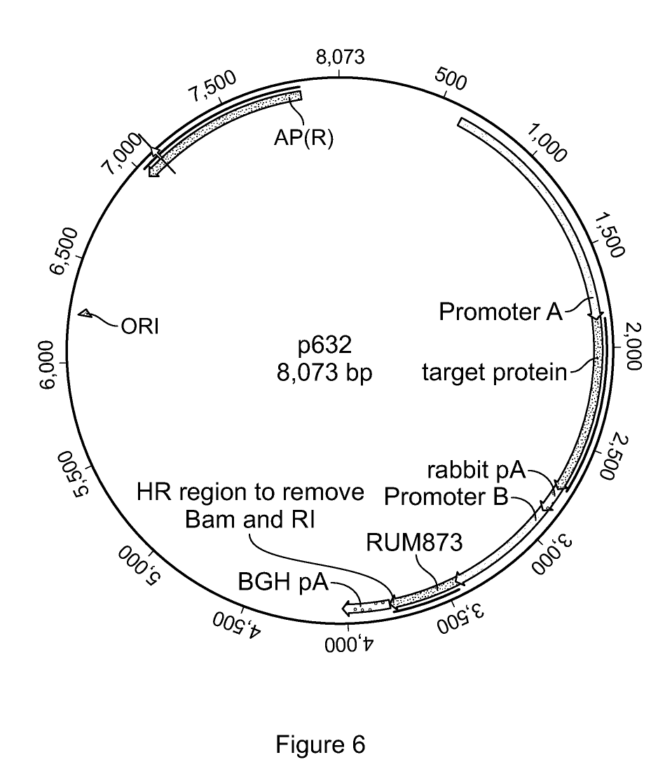

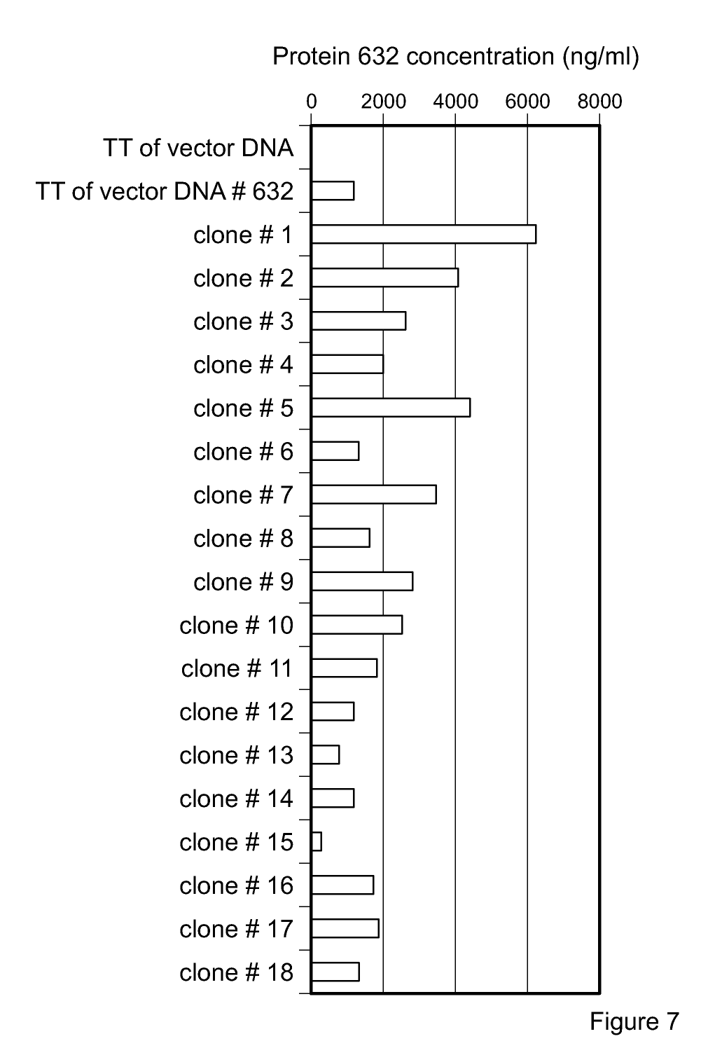

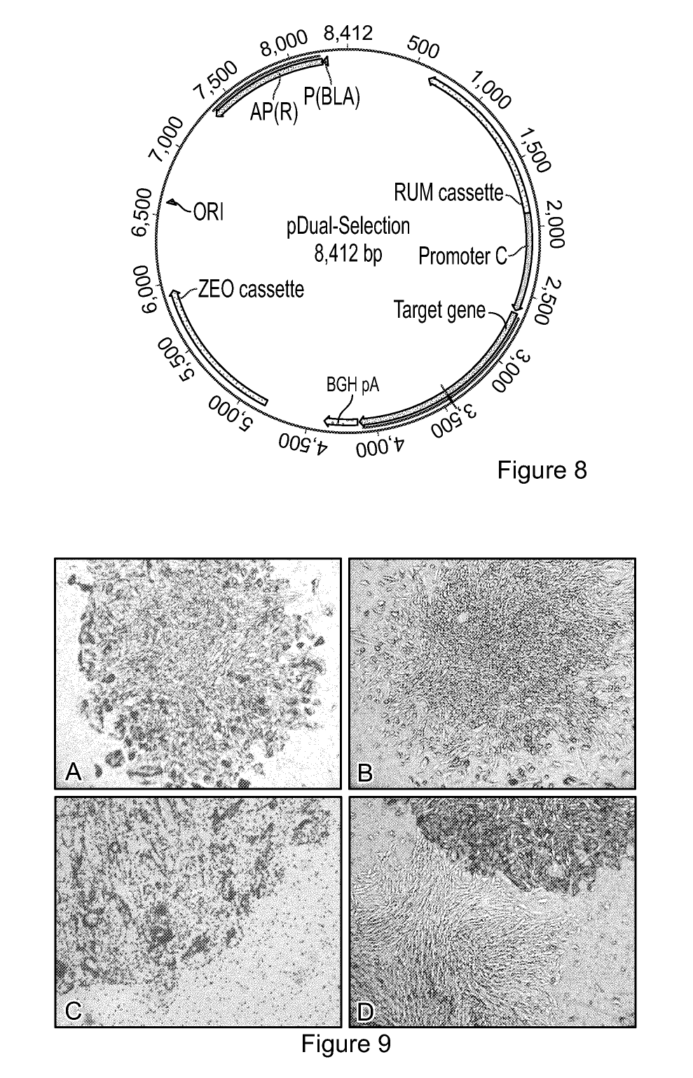

We sought to apply RUM selectable marker to generate stable cell lines expressing a target protein that can be conveniently detected. DNA vector p632 was constructed by molecular cloning to contain two expression cassettes: RUM cassette and second expression cassette expressing a target protein called #632. This target protein is a secreted molecule for which we have an ELISA that can quantify the levels of #632 in the conditioned media. Vector map of DNA vector p632 is shown in FIG. 6 (target protein is #632 in the present experiment). CHO cells were transfected with this vector DNA, and transfected cells were plated out in 10 centimeter dishes in a manner that produces single colonies after 7 days. After the colonies have appeared, they were incubated in media containing magnetic micro beads coated with an anti-FLAG antibody. After 30 minute incubation, colonies were washed 3 times with PBS, and were then inspected under a microscope. There were a number of colonies that were “stained” with significant number of magnetic beads. Some of these colonies were isolated from plates with a micropipette, and were transferred to 24-well plates. Cells were grown further, and after the presence of RUM was reexamined, there were 18 independent clones that were RUM positive. The conditioned media from these 18 cell lines were tested in the ELISA that measures the levels of the target protein, along with cells transiently transfected with either control DNA or the vector DNA p632. As shown in FIG. 7, the target protein was detected in the condition media from all 18 cell lines, and the levels are comparable to transfection of p632, while it was not detectable in CHO cells transfected with control DNA. These results demonstrated the utility of RUM as a selectable marker for stable cell line generation.

### Example 6

### FACS Analysis of RUM Positive Cells

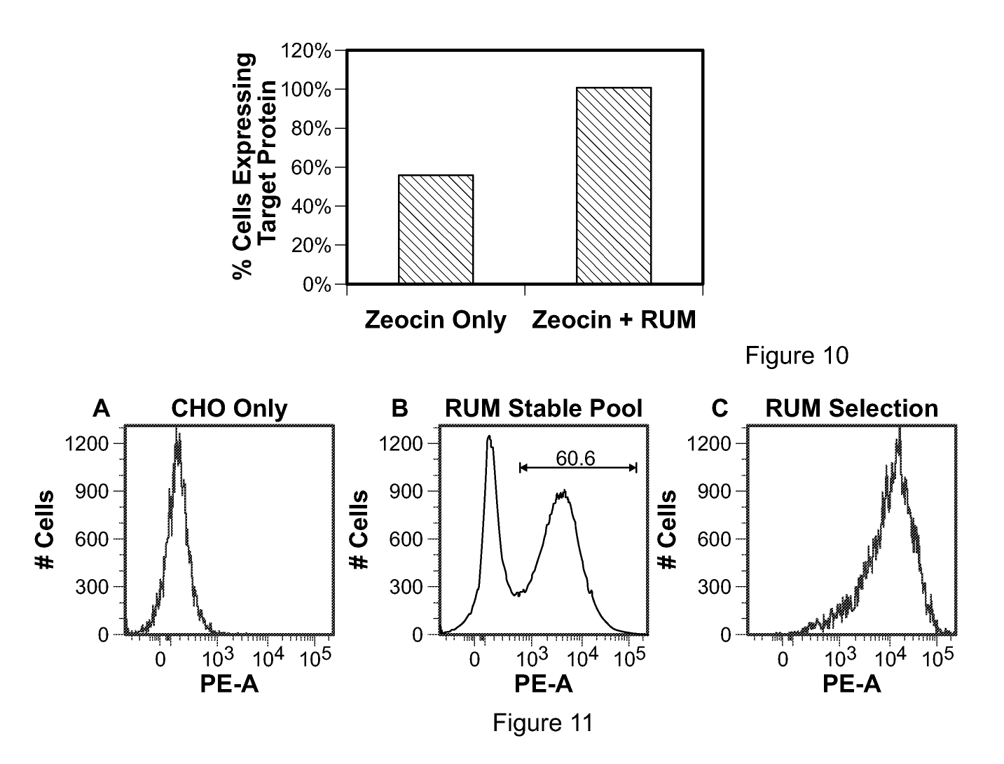

CHO cells transfected with a RUM and zeocin-containing DNA plasmid were selected as stable pools in media containing zeocin over a period of two weeks. The stable pools of cells were incubated with anti-FLAG primary antibody and then anti-mouse conjugated with R-Phycoerythrin dye secondary antibody for FACS sorting at the PE wavelength. As shown in FIG. 10B, 60.6% of the cells expressed substantial amounts of RUM on the cell surface (compare with 10A, control with CHO cells only). Transfected CHO cells expressing high levels of surface RUM were subsequently sorted by FACS to select only the best expressing cells. The FACS profile of the selected cells is shown in FIG. 10C.

### Example 7

### Generation of Stable Cells Expressing a Target Protein Using RUM as Selectable Marker

To generate a stable cell line using RUM as a selectable marker, CHO cells were transfected with a DNA construct encoding zeocin only or a construct encoding zeocin and RUM. Cells were selected in media containing zeocin and based on binding to anti-FLAG coated magnetic microbeads. Colonies of the isolated cells were grown and inspected by microscopy for the ability to bind to anti-FLAG microbeads. As shown in FIG. 11, all 20 (100%) of the zeocin+RUM stable cell line colonies inspected were bound to microbeads.

### Example 8

### Isolation of RUM Positive Cells Using Calcium-Dependent Antibody

COS7 and CHO cells are both transfected with either negative control or a plasmid DNA expression vector containing the RUM cassette at the N-terminus of the target gene in 24-well tissue culture plates. Attached cells are harvested two days after transfection and incubated with 300 μl DMEM/F12 containing 1 ug/ml of anti-FLAG antibody (M1, Sigma), to bind the RUM epitope located at the amino terminus of the target protein, and 0.01 ug/ul of Protein A-coated magnetic beads that recognize the anti-FLAG antibody. After treatment with magnetic beads, 100 μl trypsin is added to the wells to disrupt cell attachment. After 2 minutes of trypsin treatment, 200 μl of fresh DMEM/F12 media is added to each well, and the media is pipetted up and down at least 5 times to facilitate the detachment of mammalian cells. Media containing the cell suspension is then transferred to microtubes, and placed under a magnetic field in order for magnetic beads to move the side of microtubes closest to the magnetic field, and accumulate there. With the tubes firmly under the magnetic field, media is pipetted out, and cells are washed twice with fresh DMEM/F12. After the wash, the tubes are removed from the magnet, and resuspended in media containing EDTA to release the isolated RUM expressing cells from the FLAG antibody-magnetic bead complex before the cells are transferred to new tissue culture plates.

1. A method of producing a recombinant cell, comprising: exposing a population of host cells comprising: i) a first nucleic acid sequence comprising a gene of interest, and ii) a second nucleic acid sequence comprising a coding sequence for a surface marker to a separation means that recognizes the surface marker, wherein cells recognized by the separation means can be separated from the rest of the cells.

2. A method of large scale production of a gene expression product, comprising exposing a population of host cells comprising: i) a first nucleic acid sequence comprising a gene of interest, and ii) a second nucleic acid sequence comprising a coding sequence for a surface marker to a separation means that recognizes the surface marker, wherein cells recognized by the separation means can be separated from the rest of the cells.

3. A method of establishing a stable cell line, comprising exposing a population of host cells comprising: i) a first nucleic acid sequence comprising a gene of interest, and ii) a second nucleic acid sequence comprising a coding sequence for a surface marker to a separation means that recognizes the surface marker, wherein cells recognized by the separation means can be separated from the rest of the cells.

4. The method of claim 2, wherein the surface marker comprises a tag sequence and a transmembrane domain.

5. The method of claim 2, wherein the surface marker comprises a tag sequence and a membrane anchoring region.

6. The method of claim 4, wherein the tag sequence is less than about 100 amino acids.

7. The method of claim 2, wherein the separation means is a ligand specifically recognizing the surface marker.

8. The method of claim 7, wherein the ligand is attached to a magnetic bead.

9. The method of claim 7, wherein the ligand is attached to a label.

10. The method of claim 7, wherein the ligand is attached to a solid support.

11. The method of claim 2, further comprising separating cells recognized by the separation means from the rest of the cells.

12. The method of claim 2, further comprising culturing the host cells for at least one day prior to exposing the cells to a separation means.

13. The methods of claim 2, further comprising introducing the first and second nucleic acid sequences into the cells.

14. The method of claim 2, wherein the first and second nucleic acid sequences are on the same expression vector.

15. The method of claim 2, further comprising washing the cells exposed to the separation means.

16. The method of claim 2, wherein the cells further comprise a third nucleic acid sequence comprising a coding sequence for a second selectable marker that is different from the surface marker.

17. The method of claim 16, further comprising selecting cells based on the second selectable marker.

18. An expression vector comprising: i) a first cistron comprising a first promoter and a gene of interest, ii) a second cistron comprising a second promoter and a coding sequence for a surface marker, wherein the surface marker comprises a tag sequence and a transmembrane domain or a membrane anchoring region.

19. The expression vector of claim 18, further comprising a third cistron comprising a third promoter and a coding sequence for a selectable marker that is different from the surface marker.

20. The expression vector of claim 18, wherein the first and second cistrons are positioned head to tail on the expression vector.

21-26. (canceled)
